SEQUENCE LISTING (7 sequences)

SEQ ID NO: 1 - DNA, 375 bp, Escherichia coli
  E. coli enterotoxin B
atgaataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactag

SEQ ID NO: 2 - DNA, 375 bp, Escherichia coli
  E. coli enterotoxin B (porcine)
atgaataaagtaaaatgttatgttttatttacggcgttactatcctctctatatgcacacggagctccccagactattacagaactatgttcggaatatcgcaacacacaaatatatacgataaatgacaagatactatcatatacggaatcgatggcaggcaaaagagaaatggttatcattacatttaagagcggcgaaacatttcaggtcgaagtcccgggcagtcaacatatagactcccagaaaaaagccattgaaaggatgaaggacacattaagaatcacatatctgaccgagaccaaaattgataaattatgtgtatggaataataaaacccccaattcaattgcggcaatcagtatgaaaaactag

SEQ ID NO: 3 - DNA, 375 bp, Vibrio cholerae
  cholera toxin subunit B
atgattaaattaaaatttggtgttttttttacagttttactatcttcagcatatgcacatggaacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 4 - protein, 4 aa, Artificial Sequence
  Description of Artificial SequenceCys-2His-2 zinc finger found in TFIIIA
CCHH

SEQ ID NO: 5 - protein, 6 aa, Artificial Sequence
  Description of Artificial SequenceZn-2(Cys)-6 cluster found in yeast Gal4
CCCCCC

SEQ ID NO: 6 - protein, 4 aa, Artificial Sequence
  Description of Artificial SequenceCys-3His box found in retroviral nucleocapsid proteins
CCCH

SEQ ID NO: 7 - protein, 8 aa, Artificial Sequence
  Description of Artificial SequenceZn-2(Cys)-8 cluster found in nuclear hormone receptor-type proteins
CCCCCCCC

(19) United States
(12) Patent Application Publication
(10) Pub. No.: US 2008/0019988 A1
(43) Pub. Date: Jan. 24, 2008

(54) Targeting of genetic vaccine vectors

(75) Inventors: Juha Punnonen, Belmont, CA (US); Willem P.C. Stemmer, Los Gatos, CA (US); Russell Howard, Los Altos, CA (US); Phillip A. Patten, Portola Valley, CA (US)

Correspondence Address: MAXYGEN, INC.;INTELLECTUAL PROPERTY DEPARTMENT, 515 GALVESTON DRIVE, REDWOOD CITY, CA, 94063, US

(73) Assignee: Maxygen, Inc., Redwood City, CA (US)

(21) Appl. No.: 11/713,514

(22) Filed: Mar. 1, 2007

Related U.S. Application Data
(63) Continuation of application No. 10/805,054, filed on Mar. 19, 2004, now abandoned.
Application No. 10/805,054 is a continuation of application No. 09/247,886, filed on Feb. 10, 1999, now abandoned.
(60) Provisional application No. 60/074,294, filed on Feb. 11, 1998.

(51) Int. Cl. A61K 39/12; A61P 31/12; C07K 14/00; C12N 15/09

(52) U.S. Cl. 424/184.1; 435/69.3; 530/350

(57) ABSTRACT

This invention provides methods of obtaining reagents for increasing the specificity of genetic vaccines for a desired target cell or tissue type. The invention also provides delivery vehicles for use to improve genetic vaccine specificity for a target cell or tissue type.

### CROSS-REFERENCE TO RELATED APPLICATIONS

This application claims benefit of U.S. Provisional Application No. 60/074,294, filed Feb. 11, 1998, which application is incorporated herein by reference for all purposes.

### BACKGROUND OF THE INVENTION

1. Field of the Invention

This invention pertains to the field of genetic vaccines. Specifically, the invention provides methods for improving the efficacy of genetic vaccines by providing materials that facilitate targeting of a genetic vaccine to a particular tissue or cell type of interest.

2. Background

Genetic immunization represents a novel mechanism of inducing protective humoral and cellular immunity. Vectors for genetic vaccinations generally consist of DNA that includes a promoter/enhancer sequence operably linked to a gene of interest (which often encodes an antigen) and a polyadenylation/transcriptional terminator sequence. After intramuscular or intradermal injection, the gene of interest is expressed followed by recognition of the resulting protein by the cells of the immune system. Genetic immunizations provide means to induce protective immunity even in situations when the pathogens are poorly characterized or cannot be isolated or cultured in laboratory environment. Antigen is expressed in the host cell cytoplasm (for example, in muscle cells) or, by inclusion of a signal secretion sequence, is expressed on the surface of the host cell or secreted from the host cell. The antigen is processed by endogenous processes of the host cell transfected by the genetic vaccine vector. When expressed cytoplasmically, the antigen is thought to be targeted to the proteasome for proteolysis. The peptides so derived are sorted by endogenous TAP-1 and TAP-2 and transported into the lumen of the rough endoplasmic reticulum (RER), where they associate with MHC Class I molecules for eventual trafficking to the cell surface as a molecular complex of Class I, β2-microglobulin and peptide. When the antigen is released intact from transfected cells, it is thought to be taken up by endocytic pathways in APC and processed internally in them by endogenous pathways for eventual presentation on their cell surface as peptide fragments in complex with MHC Class I or II molecules.

The efficacy of genetic vaccination is often limited by inefficient uptake of genetic vaccine vectors into cells. Generally, less than 1% of the muscle or skin cells at the sites of injections express the gene of interest. Even a small improvement in the efficiency of genetic vaccine vectors to enter the cells can result in a dramatic increase in the level of immune response induced by genetic vaccination. A vector typically has to cross many barriers which can result in only a very minor fraction of the DNA ever being expressed. Limitations to immunogenicity include: loss of vector due to nucleases present in blood and tissues; inefficient entry of DNA into a cell; inefficient entry of DNA into the nucleus of the cell and preference of DNA for other compartments; lack of DNA stability in the nucleus (factor limiting nuclear stability may differ from those affecting other cellular and extracellular compartments), and, for vectors that integrate into the chromosome, the efficiency of integration and the site of integration. Moreover, for many applications of genetic vaccines, it is preferable for the genetic vaccine to enter a particular target tissue or cell.

Thus, a need exists for genetic vaccines that can be targeted to specific cell and tissue types of interest, and which exhibit an increased ability to enter the target cells. The present invention fulfills these and other needs.

### SUMMARY OF THE INVENTION

The present invention provides methods for obtaining a cell-specific binding molecule that is useful for increasing uptake or specificity of a genetic vaccine to a target cell. The methods involve: creating a library of recombinant polynucleotides that by recombining a nucleic acid that encodes a polypeptide that comprises a nucleic acid binding domain and a nucleic acid that encodes a polypeptide that comprises a cell-specific binding domain; and screening the library to identify a recombinant polynucleotide that encodes a binding molecule that can bind to a nucleic acid and to a cell-specific receptor. Target cells of particular interest include antigen-presenting and antigen-processing cells, such as muscle cells, monocytes, dendritic cells, B cells, Langerhans cells, keratinocytes, and M-cells.

In some embodiments, the methods of the invention for obtaining a cell-specific binding moiety useful for increasing uptake or specificity of a genetic vaccine to a target cell involve: (1) recombining at least first and second forms of a nucleic acid which comprises a polynucleotide that encodes a nucleic acid binding domain and at least first and second forms of a nucleic acid which comprises a cell-specific ligand that specifically binds to a protein on the surface of a cell of interest, wherein the first and second forms differ from each other in two or more nucleotides, to produce a library of recombinant binding moiety-encoding nucleic acids; (2) transfecting into a population of host cells a library of vectors, each of which comprises: a) a binding site specific for the nucleic acid binding domain and 2) a member of the library of recombinant binding moiety-encoding nucleic acids, wherein the recombinant binding moiety is expressed and binds to the binding site to form a vector-binding moiety complex; (3) lysing the host cells under conditions that do not disrupt binding of the vector-binding moiety complex; (4) contacting the vector-binding moiety complex with a target cell of interest; and (5) identifying target cells that contain a vector and isolating the optimized recombinant cell-specific binding moiety nucleic acids from these target cells.

If further optimization is desired, the methods can further involve: (6) recombining at least one optimized recombinant binding moiety-encoding nucleic acid with a further form of the polynucleotide that encodes a nucleic acid binding domain and/or a further form of the polynucleotide that encodes a cell-specific ligand, which are the same or different from the first and second forms, to produce a further library of recombinant binding moiety-encoding nucleic acids; (7) transfecting into a population of host cells a library of vectors that comprise: a) a binding site specific for the nucleic acid binding domain and 2) the recombinant binding moiety-encoding nucleic acids, wherein the recombinant binding moiety is expressed and binds to the binding site to form a vector-binding moiety complex; (8) lysing the host cells under conditions that do not disrupt binding of the vector-binding moiety complex; (9) contacting the vector-binding moiety complex with a target cell of interest and identifying target cells that contain the vector; and (10) isolating the optimized recombinant binding moiety nucleic acids from the target cells which contain the vector; and (11) repeating (6) through (10), as necessary, to obtain a further optimized cell-specific binding moiety useful for increasing uptake or specificity of a genetic vaccine vector to a target cell.

The invention also provides cell-specific recombinant binding moieties produced by expressing in a host cell an optimized recombinant binding moiety-encoding nucleic acid obtained by the methods of the invention.

In another embodiment, the invention provides genetic vaccines that include: a) an optimized recombinant binding moiety that comprises a nucleic acid binding domain and a cell-specific ligand, and b) a polynucleotide sequence that comprises a binding site, wherein the nucleic acid binding domain is capable of specifically binding to the binding site.

A further embodiment of the invention provides methods for obtaining an optimized cell-specific binding moiety useful for increasing uptake, efficacy, or specificity of a genetic vaccine for a target cell by: (1) recombining at least first and second forms of a nucleic acid that comprises a polynucleotide which encodes a non-toxic receptor binding moiety of an enterotoxin or other toxin, wherein the first and second forms differ from each other in two or more nucleotides, to produce a library of recombinant nucleic acids; (2) transfecting vectors that contain the library of nucleic acids into a population of host cells, wherein the nucleic acids are expressed to form recombinant cell-specific binding moiety polypeptides; (3) contacting the recombinant cell-specific binding moiety polypeptides with a cell surface receptor of a target cell; and (4) determining which recombinant cell-specific binding moiety polypeptides exhibit enhanced ability to bind to the target cell. Methods of enhancing uptake of a genetic vaccine vector by a target cell by coating the genetic vaccine vector with an optimized recombinant cell-specific binding moiety produced by these methods are also provided by the invention.

The present invention also provides methods for evolving a vaccine delivery vehicle, genetic vaccine vector, or a vector component to obtain an optimized delivery vehicle or component that has, or confers upon a vector, enhanced ability to enter a selected mammalian tissue upon administration to a mammal. These methods involve: (1) recombining members of a pool of polynucleotides to produce a library of recombinant polynucleotides; (2) administering to a test animal a library of replicable genetic packages, each of which comprises a member of the library of recombinant polynucleotides operably linked to a polynucleotide that encodes a display polypeptide, wherein the recombinant polynucleotide and the display polypeptide are expressed as a fusion protein which is which is displayed on the surface of the replicable genetic package; and (3) recovering replicable genetic packages that are present in the selected tissue of the test animal at a suitable time after administration, wherein recovered replicable genetic packages have enhanced ability to enter the selected mammalian tissue upon administration to the mammal. If further optimization of the delivery vehicle is desired, the methods of the invention further involve: (4) recombining a nucleic acid that comprises at least one recombinant polynucleotide obtained from a replicable genetic package recovered from the selected tissue with a further pool of polynucleotides to produce a further library of recombinant polynucleotides; (5) administering to a test animal a library of replicable genetic packages, each of which comprises a member of the further library of recombinant polynucleotides operably linked to a polynucleotide that encodes a display polypeptide, wherein the recombinant polynucleotide and the display polypeptide are expressed as a fusion protein which is which is displayed on the surface of the replicable genetic package; (6) recovering replicable genetic packages that are present in the selected tissue of the test animal at a suitable time after administration; and (7) repeating (4) through (6), as necessary, to obtain a further optimized recombinant delivery vehicle that exhibits further enhanced ability to enter a selected mammalian tissue upon administration to a mammal. Methods of administration that are of particular interest include, for example, oral, topical, and inhalation. Where the administration is intravenous, mammalian tissues of interest include, for example, lymph node and spleen.

In another embodiment, the invention provides methods for evolving a vaccine delivery vehicle, genetic vaccine vector, or a vector component to obtain an optimized delivery vehicle or component to obtain an optimized delivery vehicle or vector component that has, or confers upon a vector containing the component, enhanced specificity for antigen-presenting cells by: (1) recombining members of a pool of polynucleotides to produce a library of recombinant polynucleotides; (2) producing a library of replicable genetic packages, each of which comprises a member of the library of recombinant polynucleotides operably linked to a polynucleotide that encodes a display polypeptide, wherein the recombinant polynucleotide and the display polypeptide are expressed as a fusion protein which is which is displayed on the surface of the replicable genetic package; (3) contacting the library of recombinant replicable genetic packages with a non-APC to remove replicable genetic packages that display non-APC-specific fusion polypeptides; and (4) contacting the recombinant replicable genetic packages that did not bind to the non-APC with an APC and recovering those that bind to the APC, wherein the recovered replicable genetic packages are capable of specifically binding to APCs.

In an additional embodiment, the invention provides methods for evolving a vaccine delivery vehicle, genetic vaccine vector, or a vector component to obtain an optimized delivery vehicle or component to obtain an optimized delivery vehicle or vector component that has, or confers upon a vector containing the component, an enhanced ability to enter a target cell by: (1) recombining at least first and second forms of a nucleic acid which encodes an invasin polypeptide, wherein the first and second forms differ from each other in two or more nucleotides, to produce a library of recombinant invasin nucleic acids; (2) producing a library of recombinant bacteriophage, each of which displays on the bacteriophage surface a fusion polypeptide encoded by a chimeric gene that comprises a recombinant invasin nucleic acid operably linked to a polynucleotide that encodes a display polypeptide; (3) contacting the library of recombinant bacteriophage with a population of target cells; (4) removing unbound phage and phage which is bound to the surface of the target cells; and (5) recovering phage which are present within the target cells, wherein the recovered phage are enriched for phage that have enhanced ability to enter the target cells.

In some embodiments, the optimized recombinant genetic vaccine vectors, delivery vehicles, or vector components obtained using these methods exhibit improved ability to enter an antigen presenting cell. These methods can involve washing the cells after the transfection step to remove vectors which did not enter an antigen presenting cell; culturing the cells for a predetermined time after transfection; lysing the antigen presenting cells; and isolating the optimized recombinant genetic vaccine vector from the cell lysate. Antigen presenting cells that contain an optimized recombinant genetic vaccine vectors can be identified by, for example, detecting expression of a marker gene that is included in the vectors. In some embodiments, the genetic vaccine vector comprises a nucleotide sequence that encodes an immunogenic antigen and optimized recombinant genetic vaccine vectors are identified by: transfecting individual library members into separate cultures of antigen presenting cells; co-culturing transfected APCs with T lymphocytes obtained from the same individual as the APCs; and identifying transfected APC cultures which are capable of inducing a T lymphocyte response. The T lymphocyte response in these methods can be selected from the group consisting of increased T lymphocyte proliferation, increased T lymphocyte-mediated cytolytic activity against a target cell, and increased cytokine production. As an example, the genetic vaccine vector can be capable of inducing a T_{H}1 response as evidenced by the transfected APCs inducing a T lymphocyte response that involves one or more of proliferation, IL-2 production, and interferon-γ production.

Additional embodiments of these methods involve the use of genetic vaccine vectors or delivery vehicles that include a nucleotide sequence that encodes an antigen; optimized recombinant vaccine vectors can be identified by: injecting the library of recombinant genetic vaccine vectors into a test animal; obtaining lymphatic cells (e.g., dendritic cells) from the test animal; and recovering recombinant genetic vaccine vectors from the lymphatic cells, wherein the recovered recombinant genetic vaccine vectors exhibit improved ability to enter lymphatic cells. In some embodiments, the antigen is a cell surface antigen, and prior to isolating the optimized recombinant genetic vaccine vectors, cells that contain an optimized recombinant vector are purified by binding to an affinity reagent which selectively binds to the cell surface antigen.

The invention also provides methods of evolving a bacteriophage-derived vaccine delivery vehicle to obtain a delivery vehicle having enhanced ability to enter a target cell. These methods involve the steps of: (1) recombining at least first and second forms of a nucleic acid which encodes an invasin polypeptide, wherein the first and second forms differ from each other in two or more nucleotides, to produce a library of recombinant invasin nucleic acids; (2) producing a library of recombinant bacteriophage, each of which displays on the bacteriophage surface a fusion polypeptide encoded by a chimeric gene that comprises a recombinant invasin nucleic acid operably linked to a polynucleotide that encodes a display polypeptide; (3) contacting the library of recombinant bacteriophage with a population of target cells; (4) removing unbound phage and phage which is bound to the surface of the target cells; and (5) recovering phage which are present within the target cells, wherein the recovered phage are enriched for phage that have enhanced ability to enter the target cells. Again, if further optimization is desired, the methods can include the further steps of: (6) recombining a nucleic acid which comprises at least one recombinant invasin nucleic acid obtained from a bacteriophage which is recovered from a target cell with a further pool of polynucleotides to produce a further library of recombinant invasin polynucleotides; (7) producing a further library of recombinant bacteriophage, each of which displays on the bacteriophage surface a fusion polypeptide encoded by a chimeric gene that comprises a recombinant invasin nucleic acid operably linked to a polynucleotide that encodes a display polypeptide; (8) contacting the library of recombinant bacteriophage with a population of target cells; (9) removing unbound phage and phage which is bound to the surface of the target cells; and (10) recovering phage which are present within the target cells; and (11) repeating (6) through (10), as necessary, to obtain a further optimized recombinant delivery vehicle which exhibits further have enhanced ability to enter the target cells.

In some embodiments the methods of evolving a bacteriophage-derived vaccine delivery vehicle to obtain a delivery vehicle having enhanced ability to enter a target cell can include the additional steps of: (12) inserting into the optimized recombinant delivery vehicle a polynucleotide which encodes an antigen of interest, wherein the antigen of interest is expressed as a fusion polypeptide which comprises a second display polypeptide; (13) administering the delivery vehicle to a test animal; and (14) determining whether the delivery vehicle is capable of inducing a CTL response in the test animal. Alternatively, the following steps can be employed: (12) inserting into the optimized recombinant delivery vehicle a polynucleotide which encodes an antigen of interest, wherein the antigen of interest is expressed as a fusion polypeptide which comprises a second display polypeptide; (13) administering the delivery vehicle to a test animal; and (14) determining whether the delivery vehicle is capable of inducing neutralizing antibodies against a pathogen which comprises the antigen of interest. An example of a target cell of interest for these methods is an antigen-presenting cell.

### BRIEF DESCRIPTION OF THE DRAWINGS

FIG. 1 illustrates a strategy for obtaining and using nucleic acid binding proteins that facilitate entry of genetic vaccines, in particular, naked DNA, into target cells. Members of a library obtained by DNA shuffling are linked to a coding region of M13 protein VIII so that a fusion protein is displayed on the surface of the phage particles. Phage that efficiently enter the desired target tissue are identified, and the fusion protein is then used to coat a genetic vaccine nucleic acid.

FIG. 2 illustrates a strategy for screening of M13 libraries for desired targeting of various tissues. The particular example illustrated relates to screening for improved oral delivery, but the same principle applies to libraries given by other means, including intravenously, intramuscularly, intradermally, anally, vaginally, or topically. After delivery to a test animal, the M13 phage is recovered from the tissue of interest. The procedure can be repeated to obtain further optimization.

FIG. 3 is an alignment of nucleotide sequences encoding bacterial enterotoxins from two strains of Escherichia coli and cholera toxin B. Shown are nucleotide sequences for E. coli enterotoxin B (SEQ ID NO: 1), E. coli enterotoxin B (porcine) (SEQ ID NO: 2), and Cholera toxin subunit B (SEQ ID NO: 3).

FIG. 4A and FIG. 4B show a protocol for the generation and transfection of human dendritic cells. FIG. 4A shows the phenotype of freshly isolated monocytes (left) and cultured dendritic cells obtained by culturing the blood monocytes in the presence of IL-4 and GM-CSF for seven days. FIG. 4B shows a flow cytometry analysis of cultured dendritic cells after transfection by a plasmid that encodes GFP.

### DETAILED DESCRIPTION

### Definitions

The term “cytokine” includes, for example, interleukins, interferons, chemokines, hematopoietic growth factors, tumor necrosis factors and transforming growth factors. In general these are small molecular weight proteins that regulate maturation, activation, proliferation and differentiation of the cells of the immune system.

The term “screening” describes, in general, a process that identifies optimal antigens. Several properties of the antigen can be used in selection and screening including antigen expression, folding, stability, immunogenicity and presence of epitopes from several related antigens. Selection is a form of screening in which identification and physical separation are achieved simultaneously by expression of a selection marker, which, in some genetic circumstances, allows cells expressing the marker to survive while other cells die (or vice versa). Screening markers include, for example, luciferase, beta-galactosidase and green fluorescent protein. Selection markers include drug and toxin resistance genes, and the like. Because of limitations in studying primary immune responses in vitro, in vivo studies are particularly useful screening methods. In these studies, the antigens are first introduced to test animals, and the immune responses are subsequently studied by analyzing protective immune responses or by studying the quality or strength of the induced immune response using lymphoid cells derived from the immunized animal. Although spontaneous selection can and does occur in the course of natural evolution, in the present methods selection is performed by man.

A “exogenous DNA segment”, “heterologous sequence” or a “heterologous nucleic acid”, as used herein, is one that originates from a source foreign to the particular host cell, or, if from the same source, is modified from its original form. Thus, a heterologous gene in a host cell includes a gene that is endogenous to the particular host cell, but has been modified. Modification of a heterologous sequence in the applications described herein typically occurs through the use of DNA shuffling. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

The term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “isolated”, when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

The term “naturally-occurring” is used to describe an object that can be found in nature as distinct from being artificially produced by man. For example, a polypeptide or polynucleotide sequence that is present in an organism (including viruses, bacteria, protozoa, insects, plants or mammalian tissue) that can be isolated from a source in nature and which has not been intentionally modified by man in the laboratory is naturally-occurring.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19: 5081; Ohtsuka et al. (1985) J. Biol. Chem. 260: 2605-2608; Cassol et al. (1992); Rossolini et al. (1994) Mol. Cell. Probes 8: 91-98). The term nucleic acid is used interchangeably with gene, cDNA, and mRNA encoded by a gene.

“Nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative of the presence and/or abundance of the original gene and/or gene transcript in a sample.

A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

A specific binding affinity between two molecules, for example, a ligand and a receptor, means a preferential binding of one molecule for another in a mixture of molecules. The binding of the molecules can be considered specific if the binding affinity is about 1×10⁴ M⁻¹ to about 1×10⁶ M⁻¹ or greater.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “multivalent antigenic polypeptide” or a “recombinant multivalent antigenic polypeptide” is a non-naturally occurring polypeptide that includes amino acid sequences from more than one source polypeptide, which source polypeptide is typically a naturally occurring polypeptide. At least some of the regions of different amino acid sequences constitute epitopes that are recognized by antibodies found in a mammal that has been injected with the source polypeptide. The source polypeptides from which the different epitopes are derived are usually homologous (i.e., have the same or a similar structure and/or function), and are often from different isolates, serotypes, strains, species, of organism or from different disease states, for example.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In some embodiments, the sequences are substantially identical over the entire length of the coding regions.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments such as Southern and northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays”, Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra., for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na⁺ ion, typically about 0.01 to 1.0 M Na⁺ ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

A further indication that two nucleic acid sequences or polypeptides are substantially identical is that the polypeptide encoded by the first nucleic acid is immunologically cross reactive with, or specifically binds to, the polypeptide encoded by the second nucleic acid. Thus, a polypeptide is typically substantially identical to a second polypeptide, for example, where the two peptides differ only by conservative substitutions.

The phrase “specifically (or selectively) binds to an antibody” or “specifically (or selectively) immunoreactive with”, when referring to a protein or peptide, refers to a binding reaction which is determinative of the presence of the protein, or an epitope from the protein, in the presence of a heterogeneous population of proteins and other biologics. Thus, under designated immunoassay conditions, the specified antibodies bind to a particular protein and do not bind in a significant amount to other proteins present in the sample. The antibodies raised against a multivalent antigenic polypeptide will generally bind to the proteins from which one or more of the epitopes were obtained. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following five groups each contain amino acids that are conservative substitutions for one another:

Aliphatic: Glycine (G), Alanine (A), Valine (V), Leucine (L), Isoleucine (I);

Aromatic: Phenylalanine (F), Tyrosine (Y), Tryptophan (W);

Sulfur-containing: Methionine (M), Cysteine (C);

Basic: Arginine (R), Lysine (K), Histidine (H);

Acidic: Aspartic acid (D), Glutamic acid (E), Asparagine (N), Glutamine (Q). See also, Creighton (1984) Proteins, W.H. Freeman and Company, for additional groupings of amino acids. In addition, individual substitutions, deletions or additions which alter, add or delete a single amino acid or a small percentage of amino acids in an encoded sequence are also “conservatively modified variations”.

A “subsequence” refers to a sequence of nucleic acids or amino acids that comprise a part of a longer sequence of nucleic acids or amino acids (e.g., polypeptide) respectively.

### DESCRIPTION OF THE PREFERRED EMBODIMENTS

The present invention provides reagents for facilitating the ability of a genetic vaccine to specifically bind to and enter a target cell or tissue of interest, and methods of obtaining such agents. In particular, the invention provides methods for obtaining binding peptides and delivery vehicles that, when used in conjunction with a genetic vaccine, increase the specificity of the genetic vaccine for a particular type of target cell. The methods are also useful for obtaining genetic vaccine components that can confer a desired targeting specificity when used in conjunction with a genetic vaccine vector.

A. Creation of Recombinant Libraries

The invention involves creating recombinant libraries of polynucleotides that are then screened to identify those library members that exhibit a desired property. The recombinant libraries can be created using any of various methods.

The substrate nucleic acids used for the recombination can vary depending upon the particular application. For example, where a polynucleotide that encodes a nucleic acid binding domain or a ligand for a cell-specific receptor is to be optimized, different forms of nucleic acids that encode all or part of the nucleic acid binding domain or a ligand for a cell-specific receptor are subjected to recombination. The methods require at least two variant forms of a starting substrate. The variant forms of candidate substrates can show substantial sequence or secondary structural similarity with each other, but they should also differ in at least two positions. The initial diversity between forms can be the result of natural variation, e.g., the different variant forms (homologs) are obtained from different individuals or strains of an organism (including geographic variants) or constitute related sequences from the same organism (e.g., allelic variations). Alternatively, the initial diversity can be induced, e.g., the second variant form can be generated by error-prone transcription, such as an error-prone PCR or use of a polymerase which lacks proof-reading activity (see Liao (1990) Gene 88:107-111), of the first variant form, or, by replication of the first form in a mutator strain (mutator host cells are discussed in further detail below). The initial diversity between substrates is greatly augmented in subsequent steps of recursive sequence recombination.

Often, improvements are achieved after one round of recombination and selection. However, recursive sequence recombination can be employed to achieve still further improvements in a desired property. Sequence recombination can be achieved in many different formats and permutations of formats, as described in further detail below. These formats share some common principles. Recursive sequence recombination entails successive cycles of recombination to generate molecular diversity. That is, one creates a family of nucleic acid molecules showing some sequence identity to each other but differing in the presence of mutations. In any given cycle, recombination can occur in vivo or in vitro, intracellular or extracellular. Furthermore, diversity resulting from recombination can be augmented in any cycle by applying prior methods of mutagenesis (e.g., error-prone PCR or cassette mutagenesis) to either the substrates or products for recombination. In some instances, a new or improved property or characteristic can be achieved after only a single cycle of in vivo or in vitro recombination, as when using different, variant forms of the sequence, as homologs from different individuals or strains of an organism, or related sequences from the same organism, as allelic variations.

In a presently preferred embodiment, the recombinant libraries are prepared using DNA shuffling. The shuffling and screening or selection can be used to “evolve” individual genes, whole plasmids or viruses, multigene clusters, or even whole genomes (Stemmer (1995) Bio/Technology 13:549-553). Reiterative cycles of recombination and screening/selection can be performed to further evolve the nucleic acids of interest. Such techniques do not require the extensive analysis and computation required by conventional methods for polypeptide engineering. Shuffling allows the recombination of large numbers of mutations in a minimum number of selection cycles, in contrast to traditional, pairwise recombination events. Thus, the sequence recombination techniques described herein provide particular advantages in that they provide recombination between mutations in any or all of these, thereby providing a very fast way of exploring the manner in which different combinations of mutations can affect a desired result. In some instances, however, structural and/or functional information is available which, although not required for sequence recombination, provides opportunities for modification of the technique.

Exemplary formats and examples for sequence recombination, sometimes referred to as DNA shuffling, evolution, or molecular breeding, have been described by the present inventors and co-workers in co-pending applications U.S. patent application Ser. No. 08/198,431, filed Feb. 17, 1994, Serial No. PCT/US95/02126, filed, Feb. 17, 1995, Ser. No. 08/425,684, filed Apr. 18, 1995, Ser. No. 08/537,874, filed Oct. 30, 1995, Ser. No. 08/564,955, filed Nov. 30, 1995, Ser. No. 08/621,859, filed Mar. 25, 1996, Ser. No. 08/621,430, filed Mar. 25, 1996, Serial No. PCT/US96/05480, filed Apr. 18, 1996, Ser. No. 08/650,400, filed May 20, 1996, Ser. No. 08/675,502, filed Jul. 3, 1996, Ser. No. 08/721,824, filed Sep. 27, 1996, Serial No. PCT/US97/17300, filed Sep. 26, 1997, and Serial No. PCT/US97/24239, filed Dec. 17, 1997; Stemmer, Science 270:1510 (1995); Stemmer et al., Gene 164:49-53 (1995); Stemmer, Bio/Technology 13:549-553 (1995); Stemmer, Proc. Natl. Acad. Sci. U.S.A. 91:10747-10751 (1994); Stemmer, Nature 370:389-391 (1994); Crameri et al., Nature Medicine 2(1): 1-3 (1996); Crameri et al., Nature Biotechnology 14:315-319 (1996), each of which is incorporated by reference in its entirety for all purposes.

Other methods for obtaining recombinant polynucleotides and/or for obtaining diversity in nucleic acids used as the substrates for DNA shuffling include, for example, homologous recombination (PCT/US98/05223; Publ. No. WO98/42727); oligonucleotide-directed mutagenesis (for review see, Smith, Ann. Rev. Genet. 19: 423-462 (1985); Botstein and Shortle, Science 229: 1193-1201 (1985); Carter, Biochem. J. 237: 1-7 (1986); Kunkel, “The efficiency of oligonucleotide directed mutagenesis” in Nucleic acids & Molecular Biology, Eckstein and Lilley, eds., Springer Verlag, Berlin (1987)). Included among these methods are oligonucleotide-directed mutagenesis (Zoller and Smith, Nucl. Acids Res. 10: 6487-6500 (1982), Methods in Enzymol. 100: 468-500 (1983), and Methods in Enzymol. 154: 329-350 (1987)) phosphothioate-modified DNA mutagenesis (Taylor et al., Nucl. Acids Res. 13: 8749-8764 (1985); Taylor et al., Nucl. Acids Res. 13: 8765-8787 (1985); Nakamaye and Eckstein, Nucl. Acids Res. 14: 9679-9698 (1986); Sayers et al., Nucl. Acids Res. 16: 791-802 (1988); Sayers et al., Nucl. Acids Res. 16: 803-814 (1988)), mutagenesis using uracil-containing templates (Kunkel, Proc. Nat'l. Acad. Sci. USA 82: 488-492 (1985) and Kunkel et al., Methods in Enzymol. 154: 367-382)); mutagenesis using gapped duplex DNA (Kramer et al., Nucl. Acids Res. 12: 9441-9456 (1984); Kramer and Fritz, Methods in Enzymol. 154: 350-367 (1987); Kramer et al., Nucl. Acids Res. 16: 7207 (1988)); and Fritz et al., Nucl. Acids Res. 16: 6987-6999 (1988)). Additional suitable methods include point mismatch repair (Kramer et al., Cell 38: 879-887 (1984)), mutagenesis using repair-deficient host strains (Carter et al., Nucl. Acids Res. 13: 4431-4443 (1985); Carter, Methods in Enzymol. 154: 382-403 (1987)), deletion mutagenesis (Eghtedarzadeh and Henikoff, Nucl. Acids Res. 14: 5115 (1986)), restriction-selection and restriction-purification (Wells et al., Phil. Trans. R. Soc. Lond. A 317: 415-423 (1986)), mutagenesis by total gene synthesis (Nambiar et al., Science 223: 1299-1301 (1984); Sakamar and Khorana, Nucl. Acids Res. 14: 6361-6372 (1988); Wells et al., Gene 34: 315-323 (1985); and Grundstrom et al., Nucl. Acids Res. 13: 3305-3316 (1985). Kits for mutagenesis are commercially available (e.g., Bio-Rad, Amersham International, Anglian Biotechnology).

B. Screening Methods

A recombination cycle is usually followed by at least one cycle of screening or selection for molecules having a desired property or characteristic. If a recombination cycle is performed in vitro, the products of recombination, i.e., recombinant segments, are sometimes introduced into cells before the screening step. Recombinant segments can also be linked to an appropriate vector or other regulatory sequences before screening. Alternatively, products of recombination generated in vitro are sometimes packaged as viruses before screening. If recombination is performed in vivo, recombination products can sometimes be screened in the cells in which recombination occurred. In other applications, recombinant segments are extracted from the cells, and optionally packaged as viruses, before screening.

The nature of screening or selection depends on what property or characteristic is to be acquired or the property or characteristic for which improvement is sought, and many examples are discussed below. It is not usually necessary to understand the molecular basis by which particular products of recombination (recombinant segments) have acquired new or improved properties or characteristics relative to the starting substrates. For example, a genetic vaccine vector can have many component sequences each having a different intended role (e.g., coding sequence, regulatory sequences, targeting sequences, stability-conferring sequences, immunomodulatory sequences, sequences affecting antigen presentation, and sequences affecting integration). Each of these component sequences can be varied and recombined simultaneously. Screening/selection can then be performed, for example, for recombinant segments that have increased episomal maintenance in a target cell without the need to attribute such improvement to any of the individual component sequences of the vector.

Depending on the particular screening protocol used for a desired property, initial round(s) of screening can sometimes be performed in bacterial cells due to high transfection efficiencies and ease of culture. Later rounds, and other types of screening which are not amenable to screening in bacterial cells, are performed in mammalian cells to optimize recombinant segments for use in an environment close to that of their intended use. Final rounds of screening can be performed in the precise cell type of intended use (e.g., a human antigen-presenting cell). In some instances, this cell can be obtained from a patient to be treated with a view, for example, to minimizing problems of immunogenicity in this patient.

The screening or selection step identifies a subpopulation of recombinant segments that have evolved toward acquisition of a new or improved desired property or properties useful in genetic vaccination. Depending on the screen, the recombinant segments can be identified as components of cells, components of viruses or in free form. More than one round of screening or selection can be performed after each round of recombination.

If further improvement in a property is desired, at least one and usually a collection of recombinant segments surviving a first round of screening/selection are subject to a further round of recombination. These recombinant segments can be recombined with each other or with exogenous segments representing the original substrates or further variants thereof. Again, recombination can proceed in vitro or in vivo. If the previous screening step identifies desired recombinant segments as components of cells, the components can be subjected to further recombination in vivo, or can be subjected to further recombination in vitro, or can be isolated before performing a round of in vitro recombination. Conversely, if the previous screening step identifies desired recombinant segments in naked form or as components of viruses, these segments can be introduced into cells to perform a round of in vivo recombination. The second round of recombination, irrespective how performed, generates further recombinant segments which encompass additional diversity than is present in recombinant segments resulting from previous rounds.

The second round of recombination can be followed by a further round of screening/selection according to the principles discussed above for the first round. The stringency of screening/selection can be increased between rounds. Also, the nature of the screen and the property being screened for can vary between rounds if improvement in more than one property is desired or if acquiring more than one new property is desired. Additional rounds of recombination and screening can then be performed until the recombinant segments have sufficiently evolved to acquire the desired new or improved property or function.

Various screening methods for particular applications are described herein. In several instances, screening involves expressing the recombinant peptides or polypeptides encoded by the recombinant polynucleotides of the library as fusions with a protein that is displayed on the surface of a replicable genetic package. For example, phage display can be used. See, e.g, Cwirla et al., Proc. Natl. Acad. Sci. USA 87: 6378-6382 (1990); Devlin et al., Science 249: 404-406 (1990), Scott & Smith, Science 249: 386-388 (1990); Ladner et al., U.S. Pat. No. 5,571,698. Other replicable genetic packages include, for example, bacteria, eukaryotic viruses, yeast, and spores.

The genetic packages most frequently used for display libraries are bacteriophage, particularly filamentous phage, and especially phage M13, Fd and F1. Most work has involved inserting libraries encoding polypeptides to be displayed into either gIII or gVIII of these phage forming a fusion protein. See, e.g., Dower, WO 91/19818; Devlin, WO 91/18989; MacCafferty, WO 92/01047 (gene III); Huse, WO 92/06204; Kang, WO 92/18619 (gene VIII). Such a fusion protein comprises a signal sequence, usually but not necessarily, from the phage coat protein, a polypeptide to be displayed and either the gene III or gene VIII protein or a fragment thereof. Exogenous coding sequences are often inserted at or near the N-terminus of gene III or gene VIII although other insertion sites are possible.

Eukaryotic viruses can be used to display polypeptides in an analogous manner. For example, display of human heregulin fused to gp70 of Moloney murine leukemia virus has been reported by Han et al., Proc. Natl. Acad. Sci. USA 92: 9747-9751 (1995). Spores can also be used as replicable genetic packages. In this case, polypeptides are displayed from the outer surface of the spore. For example, spores from B. subtilis have been reported to be suitable. Sequences of coat proteins of these spores are provided by Donovan et al., J. Mol. Biol. 196, 1-10 (1987). Cells can also be used as replicable genetic packages. Polypeptides to be displayed are inserted into a gene encoding a cell protein that is expressed on the cells surface. Bacterial cells including Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli are preferred. Details of outer surface proteins are discussed by Ladner et al., U.S. Pat. No. 5,571,698 and references cited therein. For example, the lamB protein of E. coli is suitable.

A basic concept of display methods that use phage or other replicable genetic package is the establishment of a physical association between DNA encoding a polypeptide to be screened and the polypeptide. This physical association is provided by the replicable genetic package, which displays a polypeptide as part of a capsid enclosing the genome of the phage or other package, wherein the polypeptide is encoded by the genome. The establishment of a physical association between polypeptides and their genetic material allows simultaneous mass screening of very large numbers of phage bearing different polypeptides. Phage displaying a polypeptide with affinity to a target, e.g., a receptor, bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means, or the polynucleotide that encodes the peptide or polypeptide can be used as part of a genetic vaccine.

Recombinant nucleic acid libraries that are obtained by the methods described herein are screened to identify those DNA segments that have a property which is desirable for genetic vaccination. The particular screening assay employed will vary, as described below, depending on the particular property for which improvement is sought. Typically, the shuffled nucleic acid library is introduced into cells prior to screening. If the DNA shuffling format employed is an in vivo format, the library of recombinant DNA segments generated already exists in a cell. If the sequence recombination is performed in vitro, the recombinant library is preferably introduced into the desired cell type before screening/selection. The members of the recombinant library can be linked to an episome or virus before introduction or can be introduced directly.

A wide variety of cell types can be used as a recipient of evolved genes. Cells of particular interest include many bacterial cell types that are used to deliver vaccines or vaccine antigens (Courvalin et al. (1995) C. R. Acad. Sci. III 18: 1207-12), both gram-negative and gram-positive, such as salmonella (Attridge et al. (1997) Vaccine 15: 155-62), clostridium (Fox et al. (1996) Gene Ther. 3: 173-8), lactobacillus, shigella (Sizemore et al. (1995) Science 270: 299-302), E. Coli, streptococcus (Oggioni and Pozzi (1996) Gene 169: 85-90), as well as mammalian cells, including human cells. In some embodiments of the invention, the library is amplified in a first host, and is then recovered from that host and introduced to a second host more amenable to expression, selection, or screening, or any other desirable parameter. The manner in which the library is introduced into the cell type depends on the DNA-uptake characteristics of the cell type, e.g., having viral receptors, being capable of conjugation, or being naturally competent. If the cell type is unsusceptible to natural and chemical-induced competence, but susceptible to electroporation, one would usually employ electroporation. If the cell type is unsusceptible to electroporation as well, one can employ biolistics. The biolistic PDS-1000 Gene Gun (Biorad, Hercules, Calif.) uses helium pressure to accelerate DNA-coated gold or tungsten microcarriers toward target cells. The process is applicable to a wide range of tissues, including plants, bacteria, fungi, algae, intact animal tissues, tissue culture cells, and animal embryos. One can employ electronic pulse delivery, which is essentially a mild electroporation format for live tissues in animals and patients (Zhao, Advanced Drug Delivery Reviews 17:257-262 (1995)). Novel methods for making cells competent are described in International Patent Application PCT/US97/04494 (Publ. No. WO97/35957). After introduction of the library of recombinant DNA genes, the cells are optionally propagated to allow expression of genes to occur.

In many assays, a means for identifying cells that contain a particular vector is necessary. Genetic vaccine vectors of all kinds can include a selectable marker gene. Under selective conditions, only those cells that express the selectable marker will survive. Examples of suitable markers include, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l. Acad. Sci. USA 78: 2072; Southern & Berg (1982) J. Mol. Appl. Genet. 1: 327).

As an alternative to, or in addition to, a selectable marker, a genetic vaccine vector can include a screenable marker which, when expressed, confers upon a cell containing the vector a readily identifiable phenotype. For example, gene that encodes a cell surface antigen that is not normally present on the host cell is suitable. The detection means can be, for example, an antibody or other ligand which specifically binds to the cell surface antigen. Examples of suitable cell surface antigens include any CD (cluster of differentiation) antigen (CD1 to CD163) from a species other than that of the host cell which is not recognized by host-specific antibodies. Other examples include green fluorescent protein (GFP, see, e.g., Chalfie et al. (1994) Science 263:802-805; Crameri et al. (1996) Nature Biotechnol. 14: 315-319; Chalfie et al. (1995) Photochem. Photobiol. 62:651-656; Olson et al. (1995) J. Cell. Biol. 130:639-650) and related antigens, several of which are commercially available.

1. Screening for Vector Longevity or Translocation to Desired Tissue

For certain applications, it is desirable to identify those vectors with the greatest longevity as DNA, or to identify vectors which end up in tissues distant from the injection site. This can be accomplished by administering to an animal a population of recombinant genetic vaccine vectors by the chosen route of administration and, at various times thereafter excise the target tissue and recover plasmid from the tissue by standard molecular biology procedures. The recovered vector molecules can be amplified in, for example, E. coli and/or by PCR in vitro. The PCR amplification can involve further gene shuffling, after which the derived selected population used for readministration to animals and further improvement of the vector. After several rounds of this procedure, the selected plasmids can be tested for their capacity to express the antigen in the correct conformation under the same conditions as the plasmid was selected in vivo.

Because antigen expression is not part of the selection or screening process described above, not all vectors obtained are capable of expressing the desired antigen. To overcome this drawback, the invention provides methods for identifying those vectors in a genetic vaccine population that exhibit not only the desired tissue localization and longevity of DNA integrity in vivo, but retention of maximal antigen expression (or expression of other genes such as cytokines, chemokines, cell surface accessory molecules, MHC, and the like). The methods involve in vitro identification of cells which express the desired molecule using cells purified from the tissue of choice, under conditions that allow recovery of very small numbers of cells and quantitative selection of those with different levels of antigen expression as desired.

Two embodiments of the invention are described, each of which uses a library of genetic vaccine vectors as the starting point. The goal of each method is to identify those plasmids that exhibit the desired biological properties in vivo. The recombinant library represents a population of vectors that differ in known ways (e.g., a combinatorial vector library of different functional modules), or has randomly generated diversity generated either by insertion of random nucleotide stretches, or has been shuffled in vitro to introduce low level mutations across all or part of the vector.

(a) Selection for Expression of Cell Surface-Localized Antigen

In a first embodiment, the invention method involves selection for expression of cell surface-localized antigen. The antigen gene is engineered in the vaccine plasmid library such that it has a region of amino acids which is targeted to the cell membrane. For example, the region can encode a hydrophobic stretch of C-terminal amino acids which signals the attachment of a phosphoinositol-glycan (PIG) terminus on the expressed protein and directs the protein to be expressed on the surface of the transfected cell. With an antigen that is naturally a soluble protein, this method will likely not affect the three dimensional folding of the protein in this engineered fusion with a new C-terminus. With an antigen that is naturally a transmembrane protein (e.g., a surface membrane protein on pathogenic viruses, bacteria, protozoa or tumor cells) there are at least two possibilities. First, the extracellular domain can be engineered to be in fusion with the C-terminal sequence for signaling PIG-linkage. Second, the protein can be expressed in toto relying on the signalling of the host cell to direct it efficiently to the cell surface. In a minority of cases, the antigen for expression will have an endogenous PIG terminal linkage (e.g., some antigens of pathogenic protozoa).

The vector library is delivered in vivo and, after a suitable interval of time tissue and/or cells from diverse target sites in the animal are collected. Cells can be purified from the tissue using standard cell biological procedures, including the use of cell specific surface reactive monoclonal antibodies as affinity reagents. It is relatively facile to purify isolated epithelial cells from mucosal sites where epithelium may have been inoculated or myoblasts from muscle. In some embodiments, minimal physical purification is performed prior to analysis. It is sometimes desirable to identify and separate specific cell populations from various tissues, such as spleen, liver, bone marrow, lymph node, and blood. Blood cells can be fractionated readily by FACS to separate B cells, CD4⁺ or CD8⁺ T cells, dendritic cells, Langerhans cells, monocytes, and the like, using diverse fluorescent monoclonal antibody reagents.

Those cells expressing the antigen can be identified with a fluorescent monoclonal antibody specific for the C-terminal sequence on PIG-linked forms of the surface antigen. FACS analysis allows quantitative assessment of the level of expression of the correct form of the antigen on the cell population. Cells expressing the maximal level of antigen are sorted and standard molecular biology methods used to recover the plasmid DNA vaccine vector that conferred this reactivity. An alternative procedure that allows purification of all those cells expressing the antigen (and that may be useful prior to loading onto a cell sorter since antigen expressing cells may be a very small minority population), is to rosette or pan-purify the cells expressing surface antigen. Rosettes can be formed between antigen expressing cells and erythrocytes bearing covalently coupled antibody to the relevant antigen. These are readily purified by unit gravity sedimentation. Panning of the cell population over petri dishes bearing immobilized monoclonal antibody specific for the relevant antigen can also be used to remove unwanted cells.

Cells expressing the required conformational structure of the target antigen can be identified using specific conformationally-dependent monoclonal antibodies that are known to react specifically with the same structure as expressed on the target pathogen. Because one monoclonal antibody cannot define all aspects of correct folding of the target antigen, one can minimize the possibility of an antigen which reacts with high affinity to the diagnostic antibody but does not yield the correct conformation as defined by that in which the antigen is found on the surface of the target pathogen or as secreted from the target pathogen. One way to minimize this possibility is to use several monoclonal antibodies, each known to react with different conformational epitopes in the correctly folded protein, in the selection process. This can be achieved by secondary FACS sorting for example.

The enriched plasmid population that successfully expressed sufficient of the antigen in the correct body site for the desired time is then used as the starting population for another round of selection, incorporating gene shuffling to expand the diversity. In this manner, one recovers the desired biological activity encoded by plasmid from tissues in DNA vaccine-immunized animals.

This method can also provide the best in vivo selected vectors that express immune accessory molecules that one may wish to incorporate into DNA vaccine constructs. For example, if it is desired to express the accessory protein B7.1 or B7.2 in antigen-presenting-cells (APC) (to promote successful presentation of antigen to T cells) one can sort APC isolated from different tissues (at or different to the inoculation site) using commercially available monoclonal antibodies that recognize functional B7 proteins.

(b) Selection for Expression of Secreted Antigen/Cytokine/Chemokine

Another method for screening is to identify plasmids in a genetic vaccine vector population that are optimal in inducing secretion of soluble proteins that can affect the qualitative and quantitative nature of an elicited immune response. For example, one can select vectors that are optimal for inducing secretion of particular cytokines, growth factors and chemokines.

The first step in these methods is to generate vectors that are contain the members of the library of recombinant nucleic acids. These vectors can then be tested individually for in vivo efficacy. The vector library is delivered to a test animal and, after a chosen interval of time, tissue and/or cells from diverse sites on the animal are collected. Cells are purified from the tissue using standard cell biological procedures, which often include the use of cell specific surface reactive monoclonal antibodies as affinity reagents. As is the case for cell surface antigens described above, physical purification of separate cell populations can be performed prior to identification of cells which express the desired protein. For these studies, the target cells for expression of cytokines will most usually be APC or B cells or T cells rather than muscle cells or epithelial cells. In such cases FACS sorting by established methods will be preferred to separate the different cell types. The different cell types described above may also be separated into relatively pure fractions using affinity panning, resetting or magnetic bead separation with panels of existing monoclonal antibodies known to define the surface membrane phenotype of murine immune cells.

Purified cells are plated onto agar plates under conditions that maintain cell viability. Cells expressing the required conformational structure of the target antigen are identified using conformationally-dependent monoclonal antibodies that are known to react specifically with the same structure as expressed on the target pathogen. Release of the relevant soluble protein from the cells is detected by incubation with monoclonal antibody, followed by a secondary reagent that gives a macroscopic signal (gold deposition, color development, fluorescence, luminescence). Cells expressing the maximal level of antigen can be identified by visual inspection, the cell or cell colony picked and standard molecular biology methods used to recover the plasmid DNA vaccine vector that conferred this reactivity. Alternatively, flow cytometry can be used to identify and select cells harboring plasmids that induce high levels of gene expression. The enriched plasmid population that successfully expressed sufficient of the soluble factor in the correct body site for the desired time is then used as the starting population for another round of selection, incorporating gene shuffling to expand the diversity, if further improvement is desired. In this manner, one recovers the desired biological activity encoded by plasmid from tissues in DNA vaccine-immunized animals.

Several monoclonal antibodies, each known to react with different conformational epitopes in the correctly folded cytokine, chemokine or growth factor, can be used to confirm that the initial results from screening with one monoclonal antibody reagent still hold when several conformational epitopes are probed. In some cases the primary probe for functional cytokine released from the cell/cell colony in agar could be a soluble domain of the cognate receptor.

(c) Flow Cytometry

Flow cytometry provides a means to efficiently analyze the functional properties of millions of individual cells. The cells are passed through an illumination zone, where they are hit by a laser beam; the scattered light and fluorescence is analyzed by computer-linked detectors. Flow cytometry provides several advantages over other methods of analyzing cell populations. Thousands of cells can be analyzed per second, with a high degree of accuracy and sensitivity. Gating of cell populations allows multiparameter analysis of each sample. Cell size, viability, and morphology can be analyzed without the need for staining. When dyes and labeled antibodies are used, one can analyze DNA content, cell surface and intracytoplasmic proteins, and identify cell type, activation state, cell cycle stage, and detect apoptosis. Up to four colors (thus, four separate antigens stained with different fluorescent labels) and light scatter characteristics can be analyzed simultaneously (four colors requires two-laser instrument; one-laser instrument can analyze three colors). The expression levels of several genes can be analyzed simultaneously, and importantly, flow cytometry-based cell sorting (“FACS sorting”) allows selection of cells with desired phenotypes. Most of the vector module libraries, including the promoter, enhancer, intron, episomal origin of replication, expression level aspect of antigen, bacterial origin and bacterial marker, can be assayed by flow cytometry to select individual human tissue culture cells that contain the recombined nucleic acid sequences that have the greatest improvement in the desired property. Typically the selection is for high level expression of a surface antigen or surrogate marker protein, as diagrammed in Error! Reference source not found. The pool of the best individual sequences is recovered from the cells selected by flow cytometry-based sorting. An advantage of this approach is that very large numbers (>107) can be evaluated in a single vial experiment.

2. In Vitro Screening Methods

Genetic vaccine vectors and vector modules can be screened for improved vaccination properties using various in vitro testing methods that are known to those of skill in the art. For example, the optimized genetic vaccines can be tested for their effect on induction of proliferation of the particular lymphocyte type of interest, e.g., B cells, T cells, T cell lines, and T cell clones. This type of screening for improved adjuvant activity and immunostimulatory properties can be performed using, for example, human or mouse cells.

A library of genetic vaccine vectors (obtained either from shuffling of random DNA or of vectors harboring genes encoding cytokines, costimulatory molecules etc.) can be screened for cytokine production (e.g., IL-2, IL-4, IL-5, IL-6, IL-10, IL-12, IL-13, IL-15, IFN-γ, TNF-α) by B cells, T cells, monocytes/macrophages, total human PBMC, or (diluted) whole blood. Cytokines can be measured by ELISA or and cytoplasmic cytokine staining and flow cytometry (single-cell analysis). Based on the cytokine production profile, one can screen for alterations in the capacity of the vectors to direct T_{H}1/T_{H}2 differentiation (as evidenced, for example, by changes in ratios of IL-4/IFN-γ, IL-4/IL-2, IL-5/IFN-γ, IL-5/IL-2, IL-13/IFN-γ, IL-13/IL-2).

Induction of APC activation can be detected based on changes in surface expression levels of activation antigens, such as B7-1 (CD80), B7-2 (CD86), MHC class I and 11, CD14, CD23, and Fc receptors, and the like.

In some embodiments, genetic vaccine vectors are analyzed for their capacity to induce T cell activation. More specifically, spleen cells from injected mice can be isolated and the capacity of cytotoxic T lymphocytes to lyse infected, autologous target cells is studied. The spleen cells are reactivated with the specific antigen in vitro. In addition, T helper cell differentiation is analyzed by measuring proliferation or production of T_{H}1 (IL-2 and IFN-γ) and T_{H}2 (IL-4 and IL-5) cytokines by ELISA and directly in CD4⁺ T cells by cytoplasmic cytokine staining and flow cytometry.

Genetic vaccines and vaccine components can also be tested for ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays involve detection of antigen expression by the target cells. For example, FACS selection provides the most efficient method of identifying cells which produce a desired antigen on the cell surface. Another advantage of FACS selection is that one can sort for different levels of expression; sometimes-lower expression may be desired. Another method involves panning using monoclonal antibodies on a plate. This method allows large numbers of cells to be handled in a short time, but the method only selects for highest expression levels. Capture by magnetic beads coated with monoclonal antibodies provides another method of identifying cells which express a particular antigen.

Genetic vaccines and vaccine components that are directed against cancer cells can be screened for their ability to inhibit proliferation of tumor cell lines in vitro. Such assays are known in the art.

An indication of the efficacy of a genetic vaccine against, for example, cancer or an autoimmune disorder, is the degree of skin inflammation when the vector is injected into the skin of a patient or test animal. Strong inflammation is correlated with strong activation of antigen-specific T cells. Improved activation of tumor-specific T cells may lead to enhanced killing of the tumors. In case of autoantigens, one can add immunomodulators that skew the responses towards T_{H}2. Skin biopsies can be taken, enabling detailed studies of the type of immune response that occurs at the sites of each injection (in mice large numbers of injections/vectors can be analyzed)

Other suitable screening methods can involve detection of changes in expression of cytokines, chemokines, accessory molecules, and the like, by cells upon challenge by a library of genetic vaccine vectors.

3. Enhanced Entry of Genetic Vaccine Vectors into Cells

The methods involve subjecting to DNA shuffling polynucleotides which are involved in cell entry. Such polynucleotides are referred to herein as “transfer sequences” or “transfer modules.” Transfer modules can be obtained which increase transfer in a cell-specific manner, or which act in a more general manner. Because the exact sequences that affect DNA binding and transfer are not often known, DNA shuffling may be the only efficient method to improve the capacity of DNA to enter the cytoplasm and subsequently the nucleus of human cells.

The methods involve recombining at least first and second forms of a nucleic acid that comprises a transfer sequence. The first and second forms differ from each other in two or more nucleotides. Suitable substrates include, for example, transcription factor binding sites, CpG sequences, poly A, C, G, T oligonucleotides, and random DNA fragments such as, for example, genomic DNA, from human or other mammalian species. It has been suggested that cell surface proteins, such as the macrophage scavenger receptor, may act as receptors for specific DNA binding (Pisetsky (1996) Immunity 5: 303). It is not known whether these receptors recognize specific DNA sequences or whether they bind DNA in a sequence non-specific manner. However, GGGG tetrads have been shown to enhance DNA binding to cell surfaces (Id.). In addition to the DNA sequence, the three-dimensional structure of the plasmids may play a role in the capacity of these plasmids to enter cells. The DNA shuffling methods of the invention provide means for optimizing such sequences for ability to confer upon a vector the ability to enter a cell even in the absence of detailed information as to the mechanism by which this effect is achieved.

The resulting library of recombinant transfer modules are screened to identify at least one optimized recombinant transfer module that enhances the capability of a vector comprising the transfer module to enter a cell of interest. For example, vectors that include a recombinant transfer module can be contacted with a population of cells under conditions conducive to entry of the vector into the cells, after which the percentage of cells in the population which contain the nucleic acid vector is determined. Preferably, the vector will contain a selectable or screenable marker to facilitate identification of cells which contain the vector. In a preferred embodiment, clonal isolates of vectors bearing recombinant segments are used to infect separate cultures of cells. The percentage of vectors which enter cells can then be determined by, for example, counting cells expressing a marker expressed by the vectors in the course of transfection.

Typically, the recombination process is repeated by recombining at least one optimized transfer sequence with a further form of the transfer sequence to produce a further library of recombinant transfer modules. The further form can be the same or different from the first and second forms. The new library is screened to identify at least one further optimized recombinant vector module that exhibits an enhancement of the ability of a genetic vaccine vector that includes the optimized transfer module to enter a cell of interest. The recombination and rescreening process can be repeated as necessary, until a transfer module that has sufficient ability to enhance transfer is obtained. After one or more of recombination and screening, vector modules are obtained which are capable of conferring upon a nucleic acid vector the ability to enter at least about 50 percent more target cells than a control vector which does not contain the optimized module, more preferably at least about 75 percent more, and most preferably at least about 95 or 99 percent more target cells than a control vector.

Although for vaccine purposes non-integrating vectors are generally preferred, for some applications it may be desirable to use an integrating vector; for these applications DNA sequences that directly or indirectly affect the efficiency of integration can be included in the genetic vaccine vector. For integration by homologous recombination, important factors are the degree and length of homology to chromosomal sequences, as well as the frequency of such sequences in the genome (e.g., Alu repeats). The specific sequence mediating homologous recombination is also important, since integration occurs much more easily in transcriptionally active DNA. Methods and materials for constructing homologous targeting constructs are described by e.g., Mansour (1988) Nature 336:348; Bradley (1992) Bio/Technology 10:534. For nonhomologous, illegitimate and site-specific recombination, recombination is mediated by specific sites on the therapy vector which interact with cell encoded recombination proteins, e.g., Cre/Lox and Flp/Frt systems. See, e.g., Baubonis (1993) Nucleic Acids Res. 21:2025-2029, which reports that a vector including a LoxP site becomes integrated at a LoxP site in chromosomal DNA in the presence of Cre recombinase enzyme.

C. Evolution of Binding Polypeptides that Enhance Specificity and Efficiency of Genetic Vaccines

The present invention also provides methods for obtaining recombinant nucleic acids that encode polypeptides which can enhance the ability of genetic vaccines to enter target cells. Although the mechanisms involved in DNA uptake are not well understood, the methods of the invention enable one to obtain genetic vaccines that exhibit enhanced entry to cells, and to appropriate cellular compartments.

In one embodiment, the invention provides methods of enhancing the efficiency and specificity of a genetic vaccine nucleic acid uptake by a given cell type by coating the nucleic acid with an evolved protein that binds to the genetic vaccine nucleic acid, and is also capable of binding to the target cell. The vector can be contacted with the protein in vitro or in vivo. In the latter situation, the protein is expressed in cells containing the vector, optionally from a coding sequence within the vector. The nucleic acid binding proteins to be evolved usually have nucleic acid binding activity but do not necessarily have any known capacity to enhance or alter nucleic acid DNA uptake.

DNA binding proteins which can be used in these methods include, but are not limited to, transcriptional regulators, enzymes involved in DNA replication (e.g., recA) and recombination, and proteins that serve structural functions on DNA (e.g., histones, protamines). Other DNA binding proteins that can be used include the phage 434 repressor, the lambda phage cI and cro repressors, the E. coli CAP protein, myc, proteins with leucine zippers and DNA binding basic domains such as fos and jun; proteins with ‘POU’ domains such as the Drosophila paired protein; proteins with domains whose structures depend on metal ion chelation such as Cys₂His₂ zinc fingers found in TFIIIA, Zn₂(Cys)₆ clusters such as those found in yeast Gal4, the Cys₃ His box found in retroviral nucleocapsid proteins, and the Zn₂(Cys)₈ clusters found in nuclear hormone receptor-type proteins; the phage P22 Arc and Mnt repressors (see Knight et al. (1989) J. Biol. Chem. 264: 3639-3642 and Bowie & Sauer (1989) J. Biol. Chem. 264: 7596-7602. RNA binding proteins are reviewed by Burd & Dreyfuss (1994) Science 265: 615-621, and include HIV Tat and Rev.

As in other methods of the invention, evolution of DNA binding proteins toward acquisition of improved or altered uptake efficiency is effective by one or more cycles of recombination and screening. The starting substrates can be nucleic acid segments encoding natural or induced variants of one or nucleic acid binding proteins, such as those mentioned above. The nucleic acid segments can be present in vectors or in isolated form for the recombination step. Recombination can proceed through any of the formats described herein.

For screening purposes, the recombined nucleic acid segments are typically inserted into a vector, if not already present in such a vector during the recombination step. The vector generally encodes a selective marker capable of being expressed in the cell type for which uptake is desired. If the DNA binding protein being evolved recognizes a specific binding site (e.g., lacI binding protein recognizes lacO), this binding site can be included in the vector. Optionally, the vector can contain multiple binding sites in tandem.

The vectors containing different recombinant segments are transformed into host cells, usually E. coli, to allow recombinant proteins to be expressed and bind to the vector encoding their genetic material. Most cells take up only a single vector and so transformation results in a population of cells, most of which contain a single species of vector. After an appropriate period to allow for expression and binding, cells are lysed under mild conditions that do not disrupt binding of vectors to DNA binding proteins. For example, a lysis buffer of 35 mM HEPES (pH 7.5 with KOH), 0.1 mM EDTA, 100 mM Na glutamate, 5% glycerol, 0.3 mg/ml BSA, 1 mM DTT, and 0.1 mM PMSF) plus lysozyme (0.3-ml at 10 mg/ml) is suitable (see Schatz et al., U.S. Pat. No. 5,338,665). The complexes of vector and nucleic acid binding protein are then contacted with cells of the type for which improved or altered uptake is desired under conditions favoring uptake. Suitable recipient cells include the human cell types that are common targets in DNA vaccination. These cells include muscle cells, monocytes/macrophages, dendritic cells, B cells, Langerhans cells, keratinocytes, and the M-cells of the gut. Cells from mammals including, for example, human, mouse, and monkey can be used for screening. Both primary cells and cells obtained from cell lines are suitable.

After incubation, cells are plated with selection for expression of the selective marker present in the vector containing the recombinant segments. Cells expressing the marker are recovered. These cells are enriched for recombinant segments encoding nucleic acid binding proteins that enhance uptake of vectors encoding the respective recombinant segments. The recombinant segments from cells expressing the marker can then be subjected to a further round of selection. Usually, the recombinant segments are first recovered from cells, e.g., by PCR amplification or by recovery of the entire vectors. The recombinant segments can then be recombined with each other or with other sources of DNA binding protein variants to generate further recombinant segments. The further recombinant segments are screened in the same manner as before.

One example of a method to evolve an optimized nucleic acid binding domain involves the shuffling of histone genes. Histone-condensed DNA can result in increased gene transfer into cells. See, e.g., Fritz et al. (1996) Human Gene Therapy 7: 1395-1404. Thus, DNA shuffling can be used to evolve the histone protein, particularly the carboxy- and amino-terminal peptide extensions, to increase the efficiency of DNA transfer into cells. In this approach, the histone is encoded by the DNA to which it will be bound. The histone library can be constructed by, for example, 1) shuffling of many related histone genes from natural diversity, 2) addition of random or partially randomized peptide sequences at the N- and C-terminal sequences of the histone, 3) by addition of pre-selected protein-encoding regions to the N- or C-termini, such as whole cDNA libraries, nuclear protein ligand libraries, etc. These proteins can be partially randomized and linked to the histone by a library of linkers.

In a variation of the above procedure, a binding site recognized by a nucleic acid binding protein can be evolved instead of, or as well as, the nucleic acid binding protein. Nucleic acid binding sites are evolved by an analogous procedure to nucleic acid binding proteins except that the starting substrates contain variant binding sites and recombinant forms of these sites are screened as a component of a vector that also encodes a nucleic acid binding protein.

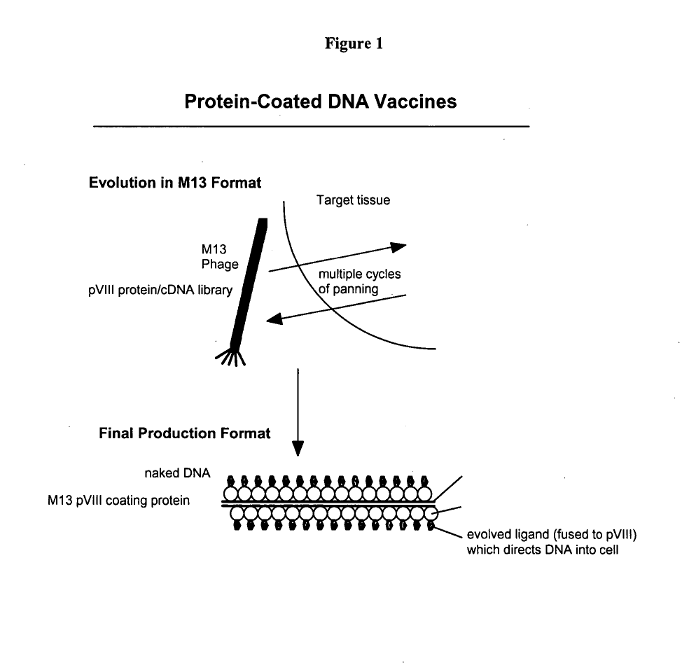

Evolved nucleic acid segments encoding DNA binding proteins and/or evolved DNA binding sites can be included in genetic vaccine vectors. If the affinity of the DNA binding protein is specific to a known DNA binding site, it is sufficient to include that binding site and the sequence encoding the DNA binding protein in the genetic vaccine vector together with such other coding and regulatory sequences are required to effect gene therapy. In some instances, the evolved DNA binding protein may not have a high degree of sequence specificity and it may be unknown precisely which sites on the vector used in screening are bound by the protein. In these circumstances, the vector should include all or most of the screening vector sequences together with additional sequences required to effect vaccination or therapy. An exemplary selection scheme which employs M13 protein VIII is shown in FIG. 1.

Target cells of interest include, for example, muscle cells, monocytes, dendritic cells, B cells, Langerhans cells, keratinocytes, M-cells of the gut, and the like. Cell-specific ligands that are suitable for use with each of the cell types are known to those of skill in the art. For example, suitable proteins to direct binding to antigen presenting cells include CD2, CD28, CTLA-4, CD40 ligand, fibrinogen, factor X, ICAM-1,0-glycan (zymosan), and the Fc portion of immunoglobulin G. (Weir's Handbook of Experimental Immunology, Eds. L. A. Herzenberg, D. M. Weir, L. A. Herzenberg, C. Blackwell, 5th edition, volume IV, chapters 156 and 174) because their respective ligands are present on APCs, including dendritic cells, monocytes/macrophages, B cells, and Langerhans cells. Bacterial enterotoxins or subunits thereof are also of interest for targeting purposes.

The ability of the vectors to enter and activate APC, such as monocytes, can also be enhanced by coating the vectors with small quantities of lipopolysaccharide (LPS). This facilitates the interaction between vector and monocytes, which have a cell surface receptor for LPS. Due to its immunostimulatory activities, LPS is also likely to act as an adjuvant, thereby further potentiating the immune responses.

Enterotoxins produced by certain pathogenic bacteria are useful as agents that bind cells and thus enhance delivery of vaccines, antigens, gene therapy vectors and pharmaceutical proteins. In an exemplary embodiment of the invention, receptor binding components of enterotoxins derived from Vibrio cholerae and enterotoxigenic strains of E. coli are evolved for improved attachment to cell surface receptors and for improved entry to and transport across the cells of the intestinal epithelium. In addition, they can be evolved for improved binding to, and activation of, B cells or other APCs. An antigen of interest can be fused to these toxin subunits to illustrate the feasibility of the approach in oral delivery of proteins and to facilitate the screening of evolved enterotoxin subunits. Examples of such antigens include growth hormone, insulin, myelin basic protein, collagen and viral envelope proteins.

These methods involve recombining at least first and second forms of a nucleic acid which comprises a polynucleotide that encodes a preferably non-toxic receptor binding moiety of an enterotoxin. The first and second forms differ from each other in two or more nucleotides, so the DNA shuffling results in production of a library of recombinant enterotoxin binding moiety nucleic acids. Suitable enterotoxins include, for example, a V. cholerae enterotoxin, enterotoxins from enterotoxigenic strains of E. coli, salmonella toxin, shigella toxin and campylobacter toxin. Vectors that contain the library of recombinant enterotoxin binding moiety nucleic acids are transfected into a population of host cells, wherein the recombinant enterotoxin binding moiety nucleic acids are expressed to form recombinant enterotoxin binding moiety polypeptides. In a preferred embodiment, the recombinant enterotoxin binding moiety polypeptides are expressed as fusion proteins on the surface of bacteriophage particles. The recombinant enterotoxin binding moiety polypeptides can be screened by contacting the library with a cell surface receptor of a target cell and determining which recombinant enterotoxin binding moiety polypeptides exhibit enhanced ability to bind to the target cell receptor. The cell surface receptor can be present on the surface of a target cell itself, or can be attached to a different cell, or binding can be tested using cell surface receptor that is not associated with a cell. Examples of suitable cell surface receptors include, for example, G_{M1}. Similarly, one can evolve bacterial superantigens for altered (increased or decreased) binding to T cell receptor and MHC class II molecules. These superantigens activate T cells in an antigen nonspecific manner. Superantigens binding to T cell receptor/MHC class II molecules include Staphylococcal enterotoxin B, Urtica dioica superantigen (Musette et al. (1996) Eur. J. Immunol. 26:618-22) and Staphylococcal enterotoxin A (Bavari et al. (1996) J. Infect. Dis. 174:338-45). Phage display has been shown to be effective when selecting superantigens that bind MHC class II molecules (Wung and Gascoigne (1997) J. Immunol. Methods. 204:33-41).

Cholera toxin (CT) is an oligomeric protein of 84,000 daltons which consists of one toxic A subunit (CT-A) covalently linked to five B subunits (CT-B). CT-B functions as the receptor binding component and binds to G_{M1} ganglioside receptors on mammalian cell surfaces. The toxic A-subunit is not necessary for the function of CT, and in the absence of CT-A, functional CT-B pentamers can form (Lebens and Holmgren (1994) Dev. Biol. Stand. 82: 215-227). Both CT and CT-B have been shown to have potent adjuvant activities in vivo and they enhance immune responses after oral delivery of antigens and vaccines (Czerkinsky et al. (1996) Ann. NY Acad. Sci. 778: 185-93; Van Cott et al. (1996) Vaccine 14: 392-8). Moreover, a single dose of CT-B conjugated to myelin basic protein prevented onset of autoimmune encephalomyelitis (EAE), a murine model of multiple sclerosis (Czerkinsky et al., supra.). Furthermore, feeding animals with myelin basic protein conjugated to CT-B after the onset of clinical symptoms (7 days) attenuated the symptoms in these animals. Other bacterial toxins, such as enterotoxins of E. coli, Salmonella toxin, Shigella toxin and Campylobacter toxin, have structural similarities with CT. Enterotoxins of E. coli have the same A-B structure as CT and they also have sequence homology and share functional similarities.

Bacterial enterotoxins can be evolved for improved affinity and entry to cells by gene shuffling. The similarity of E. coli-derived enterotoxin subunit and CT-B is 78%, and several completely conserved regions of more than eight nucleotides can be found. B subunits from two different strains of E. coli are 98% homologous both at sequence and protein levels. Thus, family DNA shuffling is feasible among enterotoxin-encoding nucleic acids from different bacterial species.

The libraries of shuffled toxin subunits can be expressed in a suitable host cell, such as V. cholerae. For safety reasons, strains in which the toxic CT-A is deleted are preferred. An antigen of interest can be fused to the receptor-binding subunit. Secretion of chimeric proteins by V. cholerae can be screened by culturing the bacteria in agar in the presence of monoclonal antibodies specific for the antigen that was fused to the toxins and the level of secretion is detected as immunoprecipitation in the agar around the colonies. One can also add G_{M1} ganglioside receptors to the agar in order to detect colonies secreting functional enterotoxin subunits. Colonies producing significant levels of the fusion protein are then cultured in 96-well plates, and the culture medium is tested for the presence of molecules capable of binding to cells or receptors in solution. Binding of chimeric fusion proteins to G_{M1} ganglioside receptors on cell surface or in solution can be detected by a monoclonal antibody specific for the antigen that was fused to the toxin. The assay using whole cells has the advantage that one may evolve for improved binding also to receptors other than the G_{M1} ganglioside receptor. When increasing concentrations of wild-type enterotoxins are added to these assays, one can detect mutants that bind to receptors with improved affinities. Affinity and specificity of toxin binding can also be determined by surface plasmon resonance (Kuziemko et al. (1996) Biochemistry 35: 6375-84).

The advantage of the bacterial expression system is that the fusion protein is secreted by bacteria that could potentially be used in large scale production. Moreover, because the fusion protein is in solution during selection, possible problems associated with expression on phage (such as bias towards selection of mutants that only function on phage) can be avoided.

Nevertheless, phage display is useful for screening to identify enterotoxins with improved affinities. A library of shuffled mutants can be expressed on phage, such as M13, and mutants with improved affinity are selected based on binding to, for example, G_{M1} ganglioside receptors in solution or on a cell surface. The advantage of this approach is that the mutants can be easily further selected in in vivo assays as discussed below. A screening approach using fusion to M13 protein VIII is diagrammed in FIG. 1.

Finally, the resulting evolved enterotoxin can be fused with DNA binding protein, and genetic vaccine vectors are coated with this fusion protein. The DNA shuffling can be done either separately, in which case the two domains are assembled after shuffling, or in a combined reaction. Shuffling results in production of a library of recombinant binding moiety nucleic acids which can be screened by transfecting vectors which contain the library, as well as a binding site specific for the nucleic acid binding domain, into a population of host cells. The binding moiety is expressed in the cells and binds to the nucleic acid binding domain to form a vector-binding moiety complex. Host cells can then be lysed under conditions that do not disrupt binding of the vector-binding moiety complex. The vector-binding moiety complex can then be contacted with a cell of interest, after which cells are identified that contain a vector and the optimized recombinant binding moiety nucleic acids are isolated from the cells.

Another method for obtaining enhanced uptake of a target DNA by mammalian cells is also provided by the invention. Specifically, the method increases the number of copies of target DNA taken into those cells that initially take up the same DNA. The method uses cell surface expression of membrane-associated DNA binding domains of, for example, transcription factors, that are encoded in the target DNA sequence, which also includes the cognate recognition sequence for the binding domain. Uptake of one molecule of target DNA into a cell (by any process, passive uptake, electroporation, osmotic shock, other stress) will lead to transcription of the gene encoding the polynucleotide binding domain. The gene encoding the binding domain is engineered so that the binding domain is expressed in a membrane anchored form. For example, a hydrophobic stretch of amino acids can be encoded at the carboxyl terminus of the binding domain, thus leading to phospho-inositol-glycan (PIG) conjugation after partial cleavage of this terminal sequence. This, in turn, leads to trafficking and positioning of the binding domain on the cell surface. The same cells that took up the first molecule of DNA will express the factor and have increased specific affinity for target DNA that remains extracellular. Cells that did not take up DNA will be at a competitive disadvantage as they will not bear the cell surface target DNA-specific binding domain, which is required for specifically mediated DNA uptake. Enhanced binding of the target DNA to the target cell will increase the efficiency of DNA internalization and desired intracellular function. This process represents a positive feedback for increased DNA uptake into cells that take up DNA first.

The target DNA, whether a circular or linear plasmid, oligonucleotide, bacterial or mammalian chromosomal fragment, is engineered to bear one or more copies of a DNA recognition sequence for a mammalian or bacterial transcription factor. Many target sequences will already bear one or more such motifs; these can be identified by sequence analysis. Endogenous motifs recognized by these factors also can be identified experimentally by demonstrating that the target DNA binds to one or more of a panel of transcription factors in an appropriate assay format. This provides a practical means for determining which factor or combination of factors to use with any particular target DNA. In the case of a small oligonucleotide or a DNA plasmid (such as used for a DNA vaccine), appropriate motifs can be engineered into the sequence. A particular motif can be engineered in one or more copies, in tandem or dispersed in the target sequence. Alternatively, a set of different motifs can be engineered, in tandem or separated, in cases where more than one DNA binding protein will be expressed on the cell surface.

D. Evolution of Bacteriophage Vectors

The invention provides methods of obtaining bacteriophage vectors that exhibit desirable properties for use as genetic vaccine vectors. The principle behind the approach provided by the invention is to combine the power of DNA shuffling with the extraordinary power of bacteriophage genetics and the wealth of recent advances in phage display technologies to rapidly evolve highly novel, potent, and generic vaccine vehicles. The evolved vaccine vehicles can present antigen either (1) in native form on the surface of these APC's for the induction of an antibody response or (2) selectively invade APC's and deliver DNA vaccine constructs to APC's for intracellular expression, processing and presentation to CTL's. More efficient methods for delivery of antigens from pathogens to professional APC's will increase the kinetics and potency of the immune response to the vaccine.

Genetic vaccine delivery vehicles that are evolved according to the methods of the invention are particularly valuable for the rapid induction of high affinity antibodies which can effectively neutralize viral epitopes or pathogenic toxins such as superantigens or cholera toxin. High affinity antibodies are generated by somatic mutation of low affinity primary response antibodies. This so-called affinity maturation process is essential for the generation of antibodies with sufficient affinity to neutralize pathogenic antigens. Affinity maturation occurs in the spleen in germinal centers where follicular dendritic cells (FDC's), professional antigen presenting cells, present protein antigens to B cells and processed antigen fragments to T cells. Clonally expanding B cell populations which have undergone somatic mutation are selected for those mutant B cells expressing antibodies with improved affinity for antigen. Thus, efficient delivery of antigen to FDC's will increase the kinetics and potency of the immune response to the immunizing antigen. Additionally, processed antigen bound to MHC is required to stimulate antigen specific T cells. Genetic vaccines are particularly efficient at priming class I MHC restricted responses due to intracellular expression of antigen, with a resultant trafficking of antigen fragments to the class I MHC pathway. Thus, invasive bacteriophage vectors capable of delivery of genetic vaccine constructs or protein antigens to FDC's are useful.

Any of several bacteriophage can be evolved according to the methods of the invention. Preferred bacteriophage for these purposes are those that have been genetically well characterized and developed for the display of foreign protein epitopes; these include, for example, lambda, T7, and M13 bacteriophage. The filamentous phage M13 is a particularly preferred vector for use in the methods of the invention. M13 is a small filamentous bacteriophage that has been used widely to display polypeptide fragments in functional, folded form on the surface of bacteriophage particles. Polypeptides have been fused to both the gene III and gene VIII coat proteins for such display purposes. Thus, M13 is a versatile, highly evolvable vehicle for efficient and targeted delivery of protein or DNA vaccine vehicles to cellular targets of interest.

The following three properties are examples of the type of improvements that can be achieved by use of the methods of the invention to evolve bacteriophage genetic vaccine vectors: (1) efficient delivery of phage to the bloodstream by inhalation or oral delivery, (2) efficient homing to APCs, and (3) efficient invasion of target cells using shuffled bacterial invasion proteins. Where M13 is used, fusions can be made to both gene III and gene VIII coat proteins so that two evolved properties can be combined into a single phage particle. These studies can be performed in test animals such as laboratory mice so that the evolved constructs can be rapidly characterized with respect to their potency as vaccine vehicles. Evolved inhalable and/or orally deliverable vehicles and evolved invasins will translate directly for use in human cells, while the principles developed in evolving the ability to home to test animal APCs are readily transferable to human cells by performing analogous selections on human APCs. While these methods are exemplified for bacteriophage vectors, the methods are also applicable to other types of genetic vaccine vectors.

(1) Evolution of Efficient Delivery of Bacteriophage Vehicles by Inhalation or Oral Delivery

The invention provides methods for obtaining genetic vaccine vectors that are capable of efficient delivery to the bloodstream upon administration by inhalation or by oral administration. Methods have been developed for the formulation of proteins into inhalable colloids that can be absorbed into the blood stream through the lung. The mechanisms by which proteins are transported into the blood stream are not clearly understood, and thus improvements are readily approached by evolutionary methods. Using M13 as an example, the invention involves preparation of a library of, for example, peptide ligands, adhesion molecules, bacterial enterotoxins, and randomly fragmented cDNA, which are fused to gene III, for example, of M13. Libraries of >1010 individual fusions are readily achievable with this technology.

Screening involves preparation of high titer stocks (preferably >10¹² phage particles) in standard colloidal formulations which are delivered intranasally to test animals, such as mice. Blood samples are taken over the course of the ensuing day and circulating phage are amplified in E. coli. It has been established that M13 circulates for long periods in the blood after injection intravenously, and thus it is reasonable to expect that phage that successfully enter the blood stream through the lung can be efficiently recovered and amplified E. coli cells. In a preferred embodiment, several rounds of enrichment are applied to the initial libraries in order to enrich for phage that can efficiently enter the blood stream when delivered intranasally. Candidate clones are typically tested individually for their relative efficiency of entry, and the best clones can be further characterized by sequencing to identify the nature of the fusions that confer efficient delivery (of particular interest from the cDNA libraries). Selected clones can be further evolved for improved entry by shuffling the entire phage genome and subjecting the phage to reiterated cycles of delivery, recovery, amplification, and shuffling.

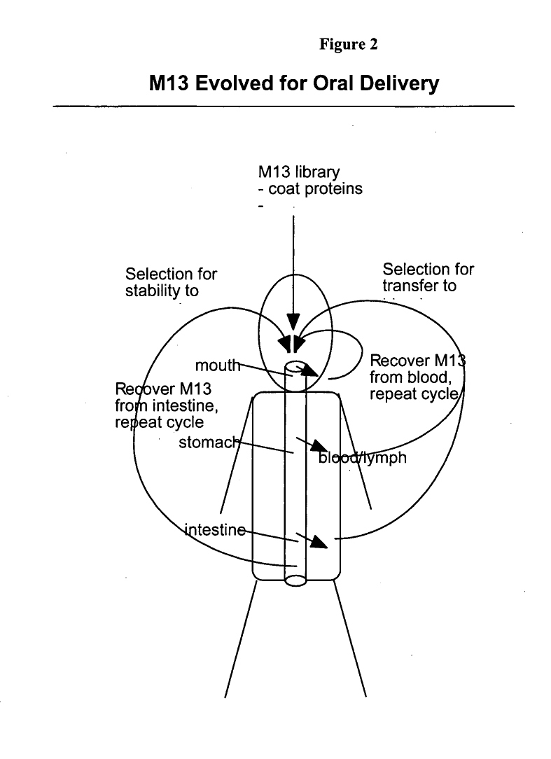

An analogous procedure is used to obtain vaccine vectors that are effective when delivered orally. A genetic vaccine vector library is prepared by DNA shuffling. The recombinant vectors are packaged and administered to a test animal. Vectors that are stable in the stomach/intestinal environment are recovered, for example, by recovering surviving vectors from the stomach. Vectors that efficiently enter the bloodstream and/or lymphatic tissue can be identified by recovering vectors that reach the blood/lymph. A schematic of this selection method is shown in FIG. 2.

(2) Evolution of Bacteriophage Vehicles for Efficient Homing to APCs

The invention also provides methods of evolving bacteriophage vectors, as well as other types of genetic vaccine vectors, for efficient homing to professional antigen presenting cells. Libraries of random peptide ligands and cDNAs used in (A) above are enriched for phage which selectively bind to APCs by first negatively selecting for binding to non-APC cell types, and then positively selecting for binding to APCs. The selections is typically performed by mixing high titer stocks of phage from the libraries (>10¹² phage particles) with cells (˜10⁷ cells per selection cycle) and either taking the nonbinding phage (negative selection) or the binding phage from cell pellets (positive selection). An alternative selection format consists of injecting phage libraries intravenously, allowing the libraries to circulate for several hours, collecting target organs of interest (lymph node, spleen), and liberating the phage by sonication. The positively selected phage can be amplified in E. coli and further rounds of enrichment are performed (3-5 rounds) if further optimization is desired. After the chosen number of rounds, individual phage are characterized for their ability to home to lymphoid organs. The best few candidates can be subjected to further evolution through iterated rounds of selection, amplification, and shuffling.

(3) Evolution of Bacteriophage for Invasion of APCs

The methods of the invention are also useful for evolving bacteriophage and other genetic vaccine vehicles for invasion of target cells. This opens up the possibility of targeting the class I MHC antigen processing pathways with either internalized protein antigen or antigen expressed by DNA vaccine vehicles carried in by the evolved vector. Invasins comprise a large family of bacterial proteins which interact with integrins and promote the efficient internalization of pathogenic bacteria such as Salmonella.

This embodiment of the invention involves shuffling different forms of polynucleotides that encode invasins. For example, two or more genes which encode the invasin family of proteins can be shuffled. The shuffled polynucleotides can be cloned as fusions to the M13 gene VIII coat protein gene, for example, and high titer stock of such libraries will be prepared. These libraries of bacteriophage can be mixed with target APCs. After incubation, the cells are exhaustively washed to remove free phage and phage bound to the surface of the cells can be removed by panning against polyclonal anti-M13 antibodies. The cells are then sonicated, thus releasing phage that have successfully entered the target cells (thus protecting them from the polyclonal anti-M13 antiserum). These phage can, if desired, be amplified, shuffled, and the selective cycle will be iteratively applied for, e.g., 3-5 times. Individual phage from the final cycle can then be characterized with respect to their relative invasiveness. The best candidates can then be combined with gene III fusions that encode pathogenic epitopes of interest. These phage can be injected into mice and tested for their relative abilities to induce a CTL response to the pathogenic antigens.

Bacteriophage vaccine vehicles evolved for activity in mice according to the above methods will establish the principles for the evolution of similar vehicles for potent human vaccines. The ability to induce more rapid and potent CTL and neutralizing antibody responses with such vehicles is an important new tool for the evolution of improved countermeasures against pathogens of interest.

Genetic Vaccine Pharmaceutical Compositions and Methods of Administration

The delivery vehicles, targeted genetic vaccine vectors, and vector components of the invention are useful for treating and/or preventing various diseases and other conditions. For example, genetic vaccines that employ the reagents obtained according to the methods of the invention are useful in both prophylaxis and therapy of infectious diseases, including those caused by any bacterial, fungal, viral, or other pathogens of mammals. The reagents obtained using the invention can also be used for treatment of autoimmune diseases including, for example, rheumatoid arthritis, SLE, diabetes mellitus, myasthenia gravis, reactive arthritis, ankylosing spondylitis, and multiple sclerosis. These and other inflammatory conditions, including IBD, psoriasis, pancreatitis, and various immunodeficiencies, can be treated using genetic vaccines that include vectors and other components obtained using the methods of the invention. Genetic vaccine vectors and other reagents obtained using the methods of the invention can be used to treat allergies and asthma. Moreover, the use of genetic vaccines have great promise for the treatment of cancer and prevention of metastasis. By inducing an immune response against cancerous cells, the body's immune system can be enlisted to reduce or eliminate cancer.

In presently preferred embodiments, the reagents obtained using the invention are used in conjunction with a genetic vaccine. The choice of vector and components can also be optimized for the particular purpose of treating allergy or other conditions. For example, an antigen for a particular condition can be optimized using recombination and selection methods analogous to those described herein. Such methods, and antigens appropriate for various conditions, are described in copending, commonly assigned U.S. patent application Ser. No. ______, entitled “Antigen Library Immunization,” which was filed on Feb. 10, 1999 as TTC Attorney Docket No. 18097-028710US. The polynucleotide that encodes the recombinant antigenic polypeptide can be placed under the control of a promoter, e.g., a high activity or tissue-specific promoter. The promoter used to express the antigenic polypeptide can itself be optimized using recombination and selection methods analogous to those described herein, as described in International Application No. PCT/US97/17300 (International Publication No. WO 98/13487). The vector can contain immunostimulatory sequences such as are described in copending, commonly assigned U.S. patent application Ser. No. ______, entitled “Optimization of Immunomodulatory Molecules,” filed as TTC Attorney Docket No. 18097-030300US on Feb. 10, 1999. The reagents obtained using the methods of the invention can also be used in conjunction with multicomponent genetic vaccines, which are capable of tailoring an immune response as is most appropriate to achieve a desired effect (see, e.g., copending, commonly assigned U.S. patent application Ser. No. ______, entitled “Genetic Vaccine Vector Engineering,” filed on Feb. 10, 1999 as TTC Attorney Docket No. 18097-030100US). It is sometimes advantageous to employ a genetic vaccine that is targeted for a particular target cell type (e.g., an antigen presenting cell or an antigen processing cell); suitable targeting methods are described in copending, commonly assigned U.S. patent application Ser. No. ______, entitled “Targeting of Genetic Vaccine Vectors,” filed on Feb. 10, 1999 as TTC Attorney Docket No. 18097-030200US.

Genetic vaccines and delivery vehicles as described herein can be delivered to a mammal (including humans) to induce a therapeutic or prophylactic immune response. Vaccine delivery vehicles can be delivered in vivo by administration to an individual patient, typically by systemic administration (e.g., intravenous, intraperitoneal, intramuscular, subdermal, intracranial, anal, vaginal, oral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; Ali et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

“Naked” DNA and/or RNA that comprises a genetic vaccine can be introduced directly into a tissue, such as muscle. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of genetic vaccines into cells of a mammal according to the invention. These methods are useful not only for in vivo introduction of DNA into a mammal, but also for ex vivo modification of cells for reintroduction into a mammal. As for other methods of delivering genetic vaccines, if necessary, vaccine administration is repeated in order to maintain the desired level of immunomodulation.

Genetic vaccine vectors (e.g., adenoviruses, liposomes, papillomaviruses, retroviruses, etc.) can be administered directly to the mammal for transduction of cells in vivo. The genetic vaccines obtained using the methods of the invention can be formulated as pharmaceutical compositions for administration in any suitable manner, including parenteral (e.g., subcutaneous, intramuscular, intradermal, or intravenous), topical, oral, rectal, intrathecal, buccal (e.g., sublingual), or local administration, such as by aerosol or transdermally, for prophylactic and/or therapeutic treatment. Pretreatment of skin, for example, by use of hair-removing agents, may be useful in transdermal delivery. Suitable methods of administering such packaged nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of genetic vaccine vector in these formulations can vary widely, and will be selected primarily based on fluid volumes, viscosities, body weight and the like in accordance with the particular mode of administration selected and the patient's needs.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the genetic vaccines, when administered orally, must be protected from digestion. This is typically accomplished either by complexing the vaccine vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient.

The packaged nucleic acids, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of packaged nucleic acid can be presented in unit-dose or multi-dose sealed containers, such as ampoules and vials.

Injection solutions and suspensions can be prepared from sterile powders, granules, and tablets of the kind previously described. Cells transduced by the packaged nucleic acid can also be administered intravenously or parenterally.

The dose administered to a patient, in the context of the present invention should be sufficient to effect a beneficial therapeutic response in the patient over time. The dose will be determined by the efficacy of the particular vector employed and the condition of the patient, as well as the body weight or vascular surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of an infection or other condition, the physician evaluates vector toxicities, progression of the disease, and the production of anti-vector antibodies, if any. In general, the dose equivalent of a naked nucleic acid from a vector is from about 1 μg to 1 mg for a typical 70 kilogram patient, and doses of vectors used to deliver the nucleic acid are calculated to yield an equivalent amount of therapeutic nucleic acid. Administration can be accomplished via single or divided doses.

In therapeutic applications, compositions are administered to a patient suffering from a disease (e.g., an infectious disease or autoimmune disorder) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the proteins of this invention to effectively treat the patient.

In prophylactic applications, compositions are administered to a human or other mammal to induce an immune response that can help protect against the establishment of an infectious disease or other condition.

The toxicity and therapeutic efficacy of the genetic vaccine vectors provided by the invention are determined using standard pharmaceutical procedures in cell cultures or experimental animals. One can determine the LD₅₀ (the dose lethal to 50% of the population) and the ED₅₀ (the dose therapeutically effective in 50% of the population) using procedures presented herein and those otherwise known to those of skill in the art.

A typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The multivalent antigenic polypeptides of the invention, and genetic vaccines that express the polypeptides, can be packaged in packs, dispenser devices, and kits for administering genetic vaccines to a mammal. For example, packs or dispenser devices that contain one or more unit dosage forms are provided. Typically, instructions for administration of the compounds will be provided with the packaging, along with a suitable indication on the label that the compound is suitable for treatment of an indicated condition. For example, the label may state that the active compound within the packaging is useful for treating a particular infectious disease, autoimmune disorder, tumor, or for preventing or treating other diseases or conditions that are mediated by, or potentially susceptible to, a mammalian immune response.

### EXAMPLES

The following examples are offered to illustrate, but not to limit the present invention.

### Example 1

### Improving the Properties of Bacterial Enterotoxins by DNA Shuffling

This Example describes the use of the DNA shuffling methods to evolve receptor binding components of enterotoxins derived from Vibrio cholerae and enterotoxigenic strains of E. coli for improved attachment to cell surface receptors and for improved entry to and transport across the cells of the intestinal epithelium. An antigen of interest can be fused to these toxin subunits to facilitate the screening of evolved enterotoxin subunits, and also to facilitate oral delivery of proteins. Examples of such antigens include growth hormone, insulin, myelin basic protein, collagen and viral envelope proteins.

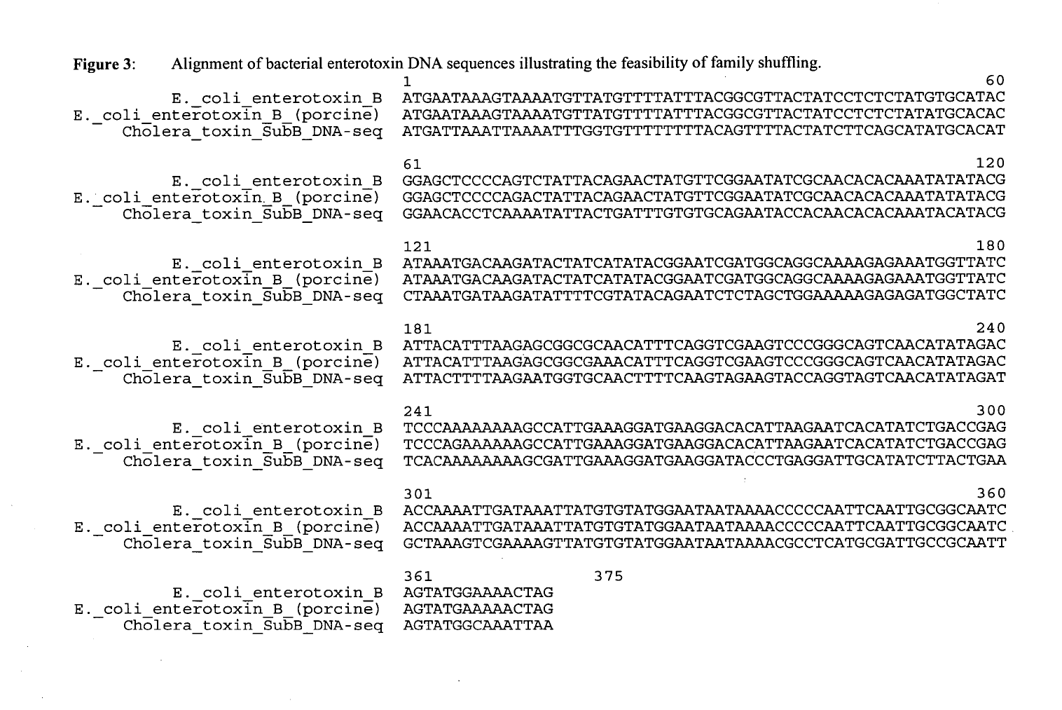

Bacterial enterotoxins are evolved for improved affinity and entry to cells by gene shuffling. The similarity of E. coli-derived enterotoxin subunit with cholera toxin CT-B is 78%, and several completely conserved regions of more than 8 nucleotides are present. An alignment of DNAs encoding CT-B and enterotoxin B subunits from two E. coli strains is shown in FIG. 3 to illustrate the feasibility of family DNA shuffling.

In one embodiment, the libraries of shuffled toxin subunits are expressed in V. cholerae. For safety reasons, strains in which the toxic CT-A is deleted are used. An antigen of interest is fused to the receptor-binding subunit. Secretion of chimeric proteins by V. cholerae can be screened by culturing the bacteria in agar in the presence of monoclonal antibodies specific for the antigen that was fused to the toxins, and detecting the level of secretion as immunoprecipitation in the agar around the colonies.

Moreover, one can also add G_{M1} ganglioside receptors to the agar in order to detect colonies secreting functional enterotoxin subunits. Colonies producing significant levels of the fusion protein are then cultured in 96-well plates, and the culture medium is tested for the presence of molecules capable of binding to cells or receptors in solution. Binding of chimeric fusion proteins to G_{M1} ganglioside receptors on cell surface or in solution can be detected by a monoclonal antibody specific for the antigen that was fused to the toxin. The assay using whole cells has the advantage that one may evolve for improved binding also to receptors other than the G_{M1} ganglioside receptor. When increasing concentrations of wild-type enterotoxins are added to these assays, one can detect mutants that bind to receptors with improved affinities.

Enterotoxins with improved affinities can also be screened using phage display methods. A library of shuffled mutants can be expressed on phage, such as M13, and mutants with improved affinity are selected based on binding to G_{M1} ganglioside receptors in solution or on cell surfaces. The advantage of this approach is that the mutants can be easily further selected in in vivo assays as discussed below.

Screening for improved oral delivery of vaccines and proteins can be done both in vitro and in vivo. The in vitro method is based on Caco-2 cells (human colon adenocarcinoma) that are cultured in tissue culture. When grown on semipermeable filters, these cells spontaneously differentiate into cells that resemble human small intestine epithelium both structurally and functionally (Hilgers et al. (1990) Pharm. Res. 7:902-910). Shuffled toxin recombinants, fused to an antigen of interest, are placed on the top of this cell layer and beneficial mutant are detected by measuring the level of antigen transport across the cell layer. Both mutants expressed in bacteria and phage can be screened using this method.

Alternatively, and additionally, the mutants are screened in vivo. When expressed on phage, a library of shuffled enterotoxin recombinants can be screened for improved entry into intestinal epithelium and blood stream after oral delivery. This screening system also allows selection of mutants with the most potent adjuvant activities. The advantage of using the phage is that a large pool of phage can be given and successful mutants can be recovered and used in succeeding rounds of shuffling and selection.

### Example 2

### Generation and Transfection of Human Dendritic Cells; Evolution of Vectors that are Optimized for these Cells

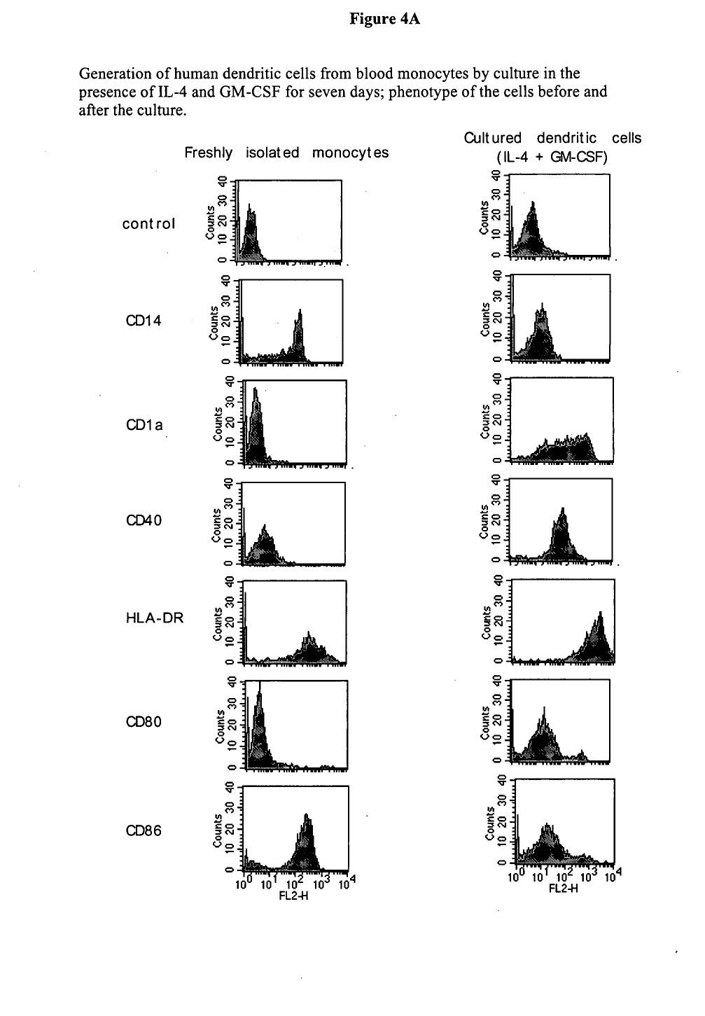

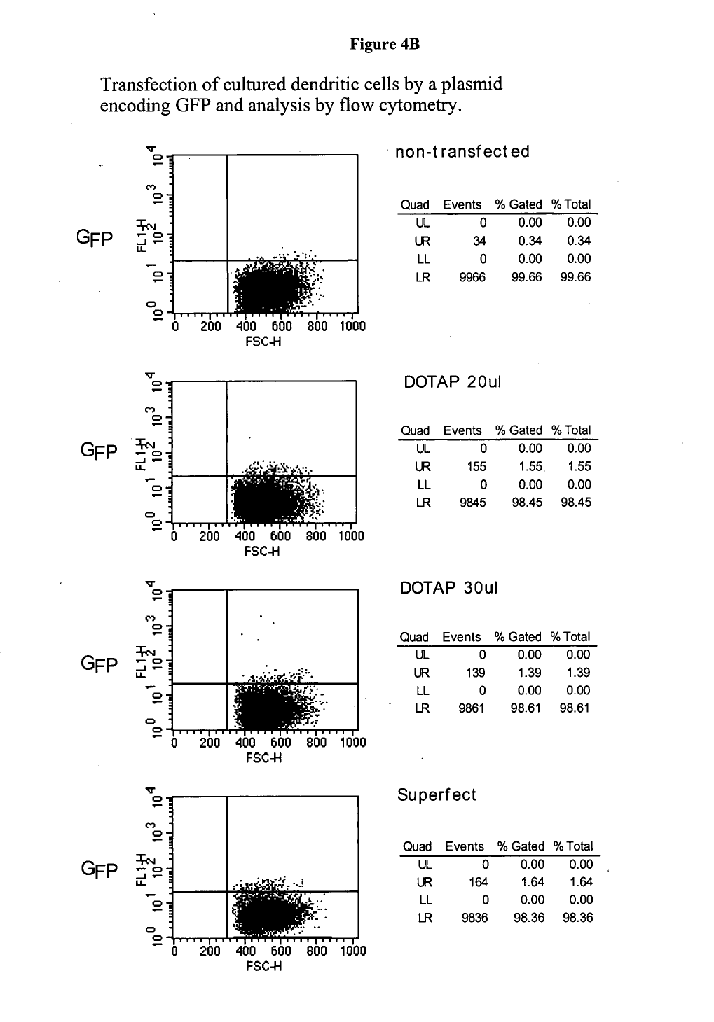

Dendritic cells are the most potent antigen presenting cells known to date. This example illustrates the feasibility of the usage of dendritic cells to screen for genetic vaccine vectors with improved properties, including transfection efficiency, expression of antigen, stability, capacity to present antigen. FIG. 4A demonstrates the phenotype of freshly isolated monocytes and after a culture period of seven days in the presence of IL-4 (400 U/ml) and GM-CSF (100 ng/ml). The cultured cells were negative for CD14, whereas they expressed CD1a, HLA-DR, CD40, CD80 and CD86, which is a characteristic phenotype of dendritic cells (Chapuis et al. (1997) Eur. J. Immunol. 27:431-441). The cultured dendritic cells were then transfected with a vector encoding GFP driven by a CMV promoter. As shown in FIG. 4B, the transfection efficiency of these cells is very low. However, a small percentage (1%) of the cells expressed low levels of GFP two days after transfection under conditions shown in the figure. These data illustrate the need for improvements in the transfection efficiency of human dendritic cells. Very little is known about the mechanisms that regulate transfection efficiency and transgene expression in dendritic cells, or how they can be improved. Therefore, DNA shuffling is an ideal approach, because it does not rely on a priori assumptions of the mechanisms that are limiting the process.

The cultured dendritic cells described in this example provide the capability to screen vector libraries described elsewhere.

### Example 3

### Selection of Bacteriophage-Derived Delivery Vehicles Having Enhanced Ability to Enter Target Cells

This Example describes a protocol for the use of phage display to select for polypeptides that can enter dendritic cells by, for example, receptor-mediated endocytosis.

A library of recombinant polynucleotides obtained by recombination of a nucleic acid binding domain and a ligand for a dendritic cell receptor is expressed in a phage display format. The phage display library is incubated with dendritic cells for a period of time, after which the cells are washed (typically multiple washes are carried out using high salt buffer) to remove phage that remain extracellular. The cells are then pelleted and sonicated to liberate phage that have been internalized. Phage that are liberated are then amplified in E. coli, and the polynucleotide that encodes the optimized recombinant binding moiety is obtained. If desired, the optimized polynucleotide is subjected to further recombination to obtain further optimization.

In a variation of this scheme, one can use a phagemid that encodes both the recombinant ligand and a selectable or screenable marker (e.g., a gene encoding green fluorescent protein operably linked to a CMV promoter). Cells that have taken up the phage can then be identified by placing the culture under selective conditions, or by methods such as fluorescence-activated cell sorting.

### Example 4

### Animal Model for Screening Genetic Vaccine Vectors

This Example provides a mouse model system that is useful for screening and testing genetic vaccine vectors in human skin in vivo. Pieces of human skin are xenotransplanted onto the back of SCID mice. Pieces of human skin can be obtained from infants undergoing circumcision, from skin removal operations due to, for example, cosmetic reasons, or from patients undergoing amputation due to, for example, accidents. These pieces are then transplanted onto the backs of C.B-17 scid/scid (SCID) mice as described by others (Deng et al. (1997) Nature Biotechnology 15: 1388-1391; Khavari et al. (1997) Adv. Clin. Res. 15:27-35; Choate and Khavari (1997) Human Gene Therapy 8:895-901).

The vector libraries are selected, for example, after topical application to the skin. However, in an analogous manner, depending on the optimal route of immunization, the evolved vectors can also be selected after i.m., i.v., i.d., oral, anal or vaginal delivery. The DNA delivered onto the skin can be in the form of a patch, in a form of a cream, in a form of naked DNA or mixture of DNA and transfection-enhancing agent (such as proteases, lipases or lipids/liposomes), and it can be applied after mechanical abrasion, after removal of the hair, or simply by adding a droplet of DNA or DNA-lipid/liposome mixture onto the skin. Similar delivery methods apply to small animals, such as mice or rat, large animals, such as cat, dog, cow, horse or monkey, as well as humans.

Suitable proteases and lipases that enhance the delivery include, but are not limited to, individuals or mixtures of the following: a protease (such as Alcalase or Savinase) with or without an alpha-amylase, a lipase (such as Lipolase) (Sarlo et al. (1997) J. Allergy Clin. Immunol. 100:480-7).

The recovery of the optimal vectors can be done from the transfected cells by, for example, PCR, or by recovering entire vectors. One can either select vectors based purely on their capacity to enter the cells or by selecting only cells that express the antigen encoded by the vector in normal mice, monkeys or SCID mice transplanted with human skin. One can use, for example, GFP as a marker gene, and after delivery detect cells that are transfected by fluorescence microscopy or flow cytometry. The positive cells can be isolated for example by flow cytometry based cell sorting. This format allows selection of vectors that optimally express antigens in and transfect human cells in vivo.

Additionally, one can screen in mice by selecting vectors that are able to induce effective immune responses after delivery onto the skin. One can select vectors that induce highest specific antibody or CTL responses, or one can select based on induction of protective immune response following challenge by the corresponding pathogen.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for obtaining a cell-specific binding molecule useful for increasing uptake or specificity of a genetic vaccine to a target cell, the method comprising:
creating a library of recombinant polynucleotides that by recombining a nucleic acid that encodes a polypeptide that comprises a nucleic acid binding domain and a nucleic acid that encodes a polypeptide that comprises a cell-specific binding domain; and
screening the library to identify a recombinant polynucleotide that encodes a binding molecule that can bind to a nucleic acid and to a cell-specific receptor.

2. A method for obtaining a cell-specific binding moiety useful for increasing uptake or specificity of a genetic vaccine to a target cell, the method comprising:
(1) recombining at least first and second forms of a nucleic acid which comprises a polynucleotide that encodes a nucleic acid binding domain and at least first and second forms of a nucleic acid which comprises a cell-specific ligand that specifically binds to a protein on the surface of a cell of interest, wherein the first and second forms differ from each other in two or more nucleotides, to produce a library of recombinant binding moiety-encoding nucleic acids;
(2) transfecting into a population of host cells a library of vectors, each of which comprises: a) a binding site specific for the nucleic acid binding domain and 2) a member of the library of recombinant binding moiety-encoding nucleic acids, wherein the recombinant binding moiety is expressed and binds to the binding site to form a vector-binding moiety complex;
(3) lysing the host cells under conditions that do not disrupt binding of the vector-binding moiety complex;
(4) contacting the vector-binding moiety complex with a target cell of interest; and
(5) identifying target cells that contain a vector and isolating the optimized recombinant cell-specific binding moiety nucleic acids from these target cells.

3. The method of claim 2, wherein the method further comprises:
(6) recombining at least one optimized recombinant binding moiety-encoding nucleic acid with a further form of the polynucleotide that encodes a nucleic acid binding domain and/or a further form of the polynucleotide that encodes a cell-specific ligand, which are the same or different from the first and second forms, to produce a further library of recombinant binding moiety-encoding nucleic acids;
(7) transfecting into a population of host cells a library of vectors that comprise: a) a binding site specific for the nucleic acid binding domain and 2) the recombinant binding moiety-encoding nucleic acids, wherein the recombinant binding moiety is expressed and binds to the binding site to form a vector-binding moiety complex;
(8) lysing the host cells under conditions that do not disrupt binding of the vector-binding moiety complex;
(9) contacting the vector-binding moiety complex with a target cell of interest and identifying target cells that contain the vector; and
(10) isolating the optimized recombinant binding moiety nucleic acids from the target cells which contain the vector; and
(11) repeating (6) through (10), as necessary, to obtain a further optimized cell-specific binding moiety useful for increasing uptake or specificity of a genetic vaccine vector to a target cell.

4. The method of claim 2, wherein the method further comprises identifying cell-specific binding moieties that result in the highest efficiency in transfecting the target cells.

5. The method of claim 2, wherein the nucleic acid binding domain is a DNA binding domain derived from a protein selected from the group consisting of a transcriptional regulator, a polypeptide involved in DNA replication or recombination, a repressor, a histone, a protamine, an E. Coli CAP protein, myc, a protein having a leucine zipper, a protein having a DNA binding basic domain, a protein having a POU domain, a protein having a zinc finger, and a protein having a Cys₃His box.

6. The method of claim 2, wherein the nucleic acid binding domain is an RNA binding domain derived from a protein selected from the group consisting of HIV tat and HIV rev.

7. The method of claim 2, wherein the target cell of interest is selected from the group consisting of muscle cells, monocytes, dendritic cells, B cells, Langerhans cells, keratinocytes, and M-cells.

8. The method of claim 7, wherein the cell of interest is a professional antigen presenting cell.

9. The method of claim 8, wherein the antigen presenting cell is a dendritic cell, a monocyte/macrophage, a B cell, or a Langerhans cell.

10. The method of claim 8, wherein the cell-specific ligand comprises a polypeptide selected from the group consisting of CD2, CD28, CTLA-4, CD40 ligand, fibrinogen, ICAM-1, Fc portion of immunoglobulin G, and a bacterial enterotoxin, or a subunit thereof.

11. The method of claim 2, wherein the target cell of interest is a human cell.

12. The method of claim 2, wherein the target cells that contain the vector are identified by selecting for expression of a selectable marker contained in the vector.

13. The method of claim 2, wherein the optimized recombinant binding moiety-encoding nucleic acid comprises a genetic vaccine vector.

14. A cell-specific recombinant binding moiety produced by expressing in a host cell an optimized recombinant binding moiety-encoding nucleic acid obtained by the method of claim 2.

15. A genetic vaccine that comprises a cell-specific recombinant binding moiety of claim 14.

16. A genetic vaccine that comprises an optimized recombinant binding moiety-encoding nucleic acid obtained by the method of claim 2.

17. A genetic vaccine that comprises:
a) an optimized recombinant binding moiety that comprises a nucleic acid binding domain and a cell-specific ligand, and
b) a polynucleotide sequence that comprises a binding site, wherein the nucleic acid binding domain is capable of specifically binding to the binding site.

18. A method for obtaining an optimized cell-specific binding moiety useful for increasing uptake, efficacy, or specificity of a genetic vaccine for a target cell, the method comprising:
(1) recombining at least first and second forms of a nucleic acid that comprises a polynucleotide which encodes a non-toxic receptor binding moiety of an enterotoxin, wherein the first and second forms differ from each other in two or more nucleotides, to produce a library of recombinant nucleic acids;
(2) transfecting vectors that contain the library of nucleic acids into a population of host cells, wherein the nucleic acids are expressed to form recombinant cell-specific binding moiety polypeptides;
(3) contacting the recombinant cell-specific binding moiety polypeptides with a cell surface receptor of a target cell; and
(4) determining which recombinant cell-specific binding moiety polypeptides exhibit enhanced ability to bind to the target cell.

19. The method of claim 18, wherein the cell surface receptor is present on the surface of a target cell.

20. The method of claim 18, wherein the cell surface receptor is G_{M1}.

21. The method of claim 18, wherein the host cell is a V cholerae cell which is incapable of expressing CT-A.

22. A method for enhancing uptake of a genetic vaccine vector by a target cell, the method comprising coating the genetic vaccine vector with an optimized recombinant cell-specific binding moiety produced by the method of claim 18.

23. The method of claim 18, wherein the recombinant cell-specific binding moieties are expressed as a fusion protein on the surface of a replicable genetic package.

24. A method of obtaining a genetic vaccine component that confers upon a vector an enhanced ability to enter an antigen-presenting cell, the method comprising:
creating a library of recombinant nucleic acids by subjecting to recombination at least two forms of a polynucleotide;
contacting a library of vectors, each of which comprises a member of the library of recombinant nucleic acids, with a population of antigen-presenting or antigen-processing cells; and
determining the percentage of cells in the population that contain the vector.

25. The method of claim 24, wherein the antigen-presenting or antigen-processing cells are selected from the group consisting of B cells, monocytes/macrophages, dendritic cells, Langerhans cells, keratinocytes, and muscle cells.

26. The method of claim 25, wherein the cells are B cells which are obtained from a B cell line.

27. The method of claim 24, wherein the screening is conducted in vivo and the cells are monkey cells or mouse cells.

28. The method of claim 24, wherein the method further comprises:
culturing the cells for a predetermined time after contacting the cells with the library of vectors;
washing the cells after the contacting step to remove vectors that did not enter an antigen-presenting cell; and
isolating the vectors from the cells that contain a vector.

29. The method of claim 24, wherein the cells that contain a vector are identified by:
transfecting individual library members or pools of library members into separate cultures of antigen-presenting cells;
co-culturing the cultures of antigen-presenting cells with T lymphocytes obtained from the same individual as the antigen-presenting cells; and
identifying cultures in which a T lymphocyte response is induced.

30. The method of claim 29, wherein the T lymphocyte response is selected from the group consisting of increased T lymphocyte proliferation, increased T lymphocyte-mediated cytolytic activity against a target cell, and increased cytokine production.

31. The method of claim 24, wherein the vector is a replicable genetic package and the recombinant nucleic acids are expressed as a fusion protein which is displayed on the surface of the replicable genetic package.

32. The method of claim 31, wherein the replicable genetic package is a bacteriophage.

33. A method of obtaining a genetic vaccine component that confers upon a vector an enhanced ability to enter cell or tissue when administered to a mammal by a desired administration protocol, the method comprising:
creating a library of recombinant nucleic acids by subjecting to recombination at least two forms of a polynucleotide;
administering to a mammal a library of vectors, each of which comprises a member of the library of recombinant nucleic acids, into a mammal;
obtaining target cells or tissues from the mammal;
identifying target cells or tissues that contain a vector, and
recovering vectors from the identified target cells or tissues.

34. The method of claim 33, wherein the target cells are lymphatic cells.

35. The method of claim 33, wherein the administering is by oral ingestion, inhalation, injection, or topical application to skin or mucous membrane.

36. The method of claim 33, wherein the vector is a replicable genetic package and the recombinant nucleic acids are expressed as a fusion protein which is displayed on the surface of the replicable genetic package.

37. A method for evolving a vaccine delivery vehicle to obtain an optimized delivery vehicle having enhanced ability to enter a selected mammalian tissue upon administration to a mammal, the method comprising:
(1) recombining members of a pool of polynucleotides to produce a library of recombinant polynucleotides;
(2) administering to a test animal a library of replicable genetic packages, each of which comprises a member of the library of recombinant polynucleotides operably linked to a polynucleotide that encodes a display polypeptide, wherein the recombinant polynucleotide and the display polypeptide are expressed as a fusion protein which is which is displayed on the surface of the replicable genetic package; and
(3) recovering replicable genetic packages that are present in the selected tissue of the test animal at a suitable time after administration, wherein recovered replicable genetic packages have enhanced ability to enter the selected mammalian tissue upon administration to the mammal.

38. The method of claim 37, wherein the method further comprises:
(4) recombining a nucleic acid that comprises at least one recombinant polynucleotide obtained from a replicable genetic package recovered from the selected tissue with a further pool of polynucleotides to produce a further library of recombinant polynucleotides;
(5) administering to a test animal a library of replicable genetic packages, each of which comprises a member of the further library of recombinant polynucleotides operably linked to a polynucleotide that encodes a display polypeptide, wherein the recombinant polynucleotide and the display polypeptide are expressed as a fusion protein which is which is displayed on the surface of the replicable genetic package;
(6) recovering replicable genetic packages that are present in the selected tissue of the test animal at a suitable time after administration; and
(7) repeating (4) through (6), as necessary, to obtain a further optimized recombinant delivery vehicle that exhibits further enhanced ability to enter a selected mammalian tissue upon administration to a mammal.

39. The method of claim 37, wherein the replicable genetic package is a bacteriophage.

40. The method of claim 39, wherein the bacteriophage is M13.

41. The method of claim 40, wherein the polynucleotide which encodes a display polypeptide is selected from the group consisting of gene III and gene VIII.

42. The method of claim 37, wherein the selected mammalian tissue is the bloodstream and the administration is by inhalation.

43. The method of claim 37, wherein the administration is intravenous and the selected mammalian tissue is selected from the group consisting of lymph node and spleen.

44. A method for evolving a vaccine delivery vehicle to obtain an optimized delivery vehicle having enhanced specificity for antigen-presenting cells, the method comprising:
(1) recombining members of a pool of polynucleotides to produce a library of recombinant polynucleotides;
(2) producing a library of replicable genetic packages, each of which comprises a member of the library of recombinant polynucleotides operably linked to a polynucleotide that encodes a display polypeptide, wherein the recombinant polynucleotide and the display polypeptide are expressed as a fusion protein which is which is displayed on the surface of the replicable genetic package;
(3) contacting the library of recombinant replicable genetic packages with a non-APC to remove replicable genetic packages that display non-APC-specific fusion polypeptides; and
(4) contacting the recombinant replicable genetic packages that did not bind to the non-APC with an APC and recovering those that bind to the APC, wherein the recovered replicable genetic packages are capable of specifically binding to APCs.

45. The method of claim 44, wherein the method further comprises the steps of:
(5) recombining a nucleic acid which comprises at least one recombinant polynucleotide obtained from a replicable genetic package that is capable of specifically binding to APCs with a further pool of polynucleotides to produce a further library of recombinant polynucleotides;
(6) producing a further library of recombinant replicable genetic packages, each of which comprises a member of the library of recombinant polynucleotides operably linked to a polynucleotide that encodes a display polypeptide, wherein the recombinant polynucleotide and the display polypeptide are expressed as a fusion protein which is which is displayed on the surface of the replicable genetic package;
(7) contacting the further library of recombinant replicable genetic packages with a non-APC to remove those that display non-APC-specific fusion polypeptides; and
(8) contacting the recombinant replicable genetic packages which did not bind to the non-APC with an APC and recovering replicable genetic packages which bind to the APC, wherein the recovered replicable genetic packages are capable of specifically binding to APCs; and
(9) repeating (5) through (8), as necessary, to obtain a further optimized recombinant delivery vehicle which exhibits further enhanced specificity for antigen-presenting cells.

46. A method for evolving a vaccine delivery vehicle to obtain an optimized delivery vehicle having enhanced ability to enter a target cell, the method comprising:
(1) recombining at least first and second forms of a nucleic acid which encodes an invasin polypeptide, wherein the first and second forms differ from each other in two or more nucleotides, to produce a library of recombinant invasin nucleic acids;
(2) producing a library of recombinant bacteriophage, each of which displays on the bacteriophage surface a fusion polypeptide encoded by a chimeric gene that comprises a recombinant invasin nucleic acid operably linked to a polynucleotide that encodes a display polypeptide;
(3) contacting the library of recombinant bacteriophage with a population of target cells;
(4) removing unbound phage and phage which is bound to the surface of the target cells; and
(5) recovering phage which are present within the target cells, wherein the recovered phage are enriched for phage that have enhanced ability to enter the target cells.

47. The method of claim 46, wherein the method further comprises:
(6) recombining a nucleic acid which comprises at least one recombinant invasin nucleic acid obtained from a bacteriophage which is recovered from a target cell with a further pool of polynucleotides to produce a further library of recombinant invasin polynucleotides;
(7) producing a further library of recombinant bacteriophage, each of which displays on the bacteriophage surface a fusion polypeptide encoded by a chimeric gene that comprises a recombinant invasin nucleic acid operably linked to a polynucleotide that encodes a display polypeptide;
(8) contacting the library of recombinant bacteriophage with a population of target cells;
(9) removing unbound phage and phage which is bound to the surface of the target cells; and
(10) recovering phage which are present within the target cells; and
(11) repeating (6) through (10), as necessary, to obtain a further optimized recombinant delivery vehicle which exhibits further have enhanced ability to enter the target cells.

48. The method of claim 47, wherein the method further comprises:
(12) inserting into the optimized recombinant delivery vehicle a polynucleotide which encodes an antigen of interest, wherein the antigen of interest is expressed as a fusion polypeptide which comprises a second display polypeptide;
(13) administering the delivery vehicle to a test animal; and
(14) determining whether the delivery vehicle is capable of inducing a CTL response in the test animal.

49. The method of claim 47, wherein the method further comprises:
(12) inserting into the optimized recombinant delivery vehicle a polynucleotide which encodes an antigen of interest, wherein the antigen of interest is expressed as a fusion polypeptide which comprises a second display polypeptide;
(13) administering the delivery vehicle to a test animal; and
(14) determining whether the delivery vehicle is capable of inducing neutralizing antibodies against a pathogen which comprises the antigen of interest.

50. The method of claim 46, wherein the target cell is an APC.
